(19) United States
(12) Patent Application Publication
(10) Pub. No.: US 2021/0401827 A1
(43) Pub. Date: Dec. 30, 2021

(54) METHODS, PARENTERAL PHARMACEUTICAL FORMULATIONS, AND DEVICES FOR THE PREVENTION OF OPIOID OVERDOSE

(71) Applicant: OPIANT PHARMACEUTICALS, INC., Santa Monica, CA (US)

(72) Inventors: Roger Crystal, Santa Monica, CA (US); Phil Skolnick, Santa Monica, CA (US)

(21) Appl. No.: 17/291,979

(22) PCT Filed: Nov. 7, 2019
(86) PCT No.: PCT/US2019/060185

§ 371 Date: May 6, 2021

Related U.S. Application Data
(60) Provisional application No. 62/756,903, filed on Nov. 7, 2018.

(51) Int. Cl. A61K 31/485; A61K 9/00; A61P 25/36; A61K 9/08; A61K 47/02

(52) U.S. Cl. CPC A61K 31/485; A61K 9/0019; A61K 47/02; A61K 9/08; A61P 25/36

(57) ABSTRACT

Methods, pharmaceutical formulations and devices for the preventative treatment of incidental opioid overdose comprising the intramuscular or subcutaneous administration using an auto-injection device of a pharmaceutical formulation containing the opioid antagonist nalmefene as a prophylactic measure.

This application claims the benefit of priority of U.S. Provisional Application No. 62/756,903, filed on Nov. 7, 2018, the disclosure of which is hereby incorporated by reference as if written herein in its entirety.

Disclosed herein are methods, formulations, and devices for the preventative treatment of incidental opioid overdose comprising the parenteral administration, including self-administration, of a pharmaceutical formulation containing an opioid antagonist; e.g. naloxone, naltrexone, or nalmefene as a prophylactic measure.

Over the past 20 years, there has been an alarming rise in the misuse and abuse of prescription opioids in the United States. Among the most prominent manifestations of what is often referred to as the ‘opioid epidemic’ are a rising number of overdose deaths (estimated at more than 33,000 in 2015) and hospital visits (estimated at more than 1.25 million) linked to opioid misuse. There has been a dramatic shift in the complexion of the opioid epidemic during the past 3-5 years: an increased availability of illicit, high potency opioids exemplified by fentanyl. Thus, between 2015 and 2016, the number of opioid overdose deaths in the United Sates rose to over 42,000; fatalities attributed to fentanyl and fentanyl analogs (collectively termed “synthetic opioids”) are largely response for this dramatic spike, and now surpass both prescription opioids and heroin as the leading cause of overdose deaths.

There are multiple factors contributing to the dangers posed by fentanyl and related synthetic opioids. Thus, unlike naturally occurring; e.g. morphine and semi-synthetic opiates; e.g. heroin and oxycodone, these synthetic opioids (hereafter referred to as “synthetics”) are piperidine derivatives. The chemistry of synthetics like fentanyl is simple compared to morphinans: fentanyl contains no centers of asymmetry, and its structure is highly conducive to derivatization. Synthetics are relatively inexpensive to produce (the cost of a kg of illicit fentanyl is roughly $3,500 compared to $65,000 for heroin, Frank, R and Pollack, H. 2017. NEJM 376:605-607) and fentanyl is ˜50-fold more potent than heroin Volkow, N D and Collins, F. 2017. NEJM 377:391-394. Multiple fentanyl derivatives (4-fluoroisobutyrlfentanyl, acrylfentanyl, acetylfentanyl, and 3-methylfentanyl), including the veterinary anesthetic, carfentanil (˜100 times more potent than fentanyl, Volkow, N D and Collins, F. 2017. NEJM 377:391-394), have been identified by the DEA in drug seizures. The potency of these illicit synthetics facilitates transport: for example, 1 kg fentanyl is equivalent to 50 kg of heroin. The lethal dose of fentanyl is ˜2 mg, suggesting a lethal dose of carfentanil is ˜20 μg, which is no more than a few specks of compound.

Fentanyl and its derivatives are absorbed through the skin, mucous membranes, and lungs. The danger posed by these synthetics is so great that guidelines have been issued to prevent occupational exposure to emergency responders and there are multiple reports of incidental contact with fentanyl by law enforcement officials resulting in hospitalization. Moreover, there is evidence that synthetics can be weaponized. In 2002, Russian security forces aerosolized a fentanyl derivative (likely a mixture of carfentanil and remifentanil) to immobilize Chechen terrorists who held hostages in a Moscow theater. The aerosolized gas resulted in the deaths of about 120 hostages. Multiple countries have assessed carfentanil and related synthetics for offensive and defensive applications, and there is a concern that terrorist groups, like ISIS, can readily obtain kg quantities of these molecules. Weaponization of high potency synthetics poses a threat to both civilians and military personnel; intentional exposure to these agents by hostile actors could incapacitate and result in fatalities.

Not only are the high potencies of synthetics problematic, but the long half-lives of fentanyl and several fentanyl derivatives; e.g. fentanyl, 8-10 h; carfentanil, 7.7 h further complicates medical management of overdose. Thus, the relatively short half-life (1-2 h) of naloxone (currently the only opioid antagonist approved to treat overdose) may require multiple doses over time in order to prevent relapse if the victim has been exposed to a long-acting opioid such as fentanyl.

There is a need for an opioid antagonist available in a form that can be administered quickly and easily as a prophylactic measure by first responders, law enforcement; e.g. police, customs, and border patrol agents and military personnel if contact with opioids, especially high potency synthetics, is either anticipated or suspected. An individual could self-administer such an agent as a preventive treatment to block or diminish the effects of incidental exposure to opioids, especially synthetic opioids, that could otherwise be fatal or lead to serious injury; e.g. hypoxic organ damage, requiring aggressive medical intervention.

Many medications can be safely and effectively self-administered either as an intramuscular or subcutaneous injection with little or no prior training using devices generally referred to as “auto-injectors”. Such devices are well known to those of ordinary skill in the art. These devices are commonly used to administer a variety of medications, ranging from proteins; e.g. insulin, to low molecular weight molecules; e.g. epinephrine, naloxone.

Opioid antagonists such as naloxone, naltrexone, and nalmefene interact at (bind to) the same brain receptors as opioids. Opioid antagonists bind to these receptors with high affinity, and compete with opioids; e.g., oxycodone, morphine, and heroin by mass action for these receptor sites. By binding to these receptors in place of opioids, opioid antagonists can reverse the pharmacological actions of opioids, including symptoms associated with overdose such as respiratory depression and somnolence.

There remains a need for durable, easy-to-use, device with storage-stable formulations that would enable untrained individuals to quickly self-administer a therapeutically effective amount of an opioid antagonist as a prophylactic measure when incidental exposure to opioids is either anticipated or suspected. This opioid antagonist should have a rapid onset to enable prophylactic use within minutes of an anticipated or suspected exposure. A long-lasting opioid antagonist would reduce the need for either a second dose of opioid antagonist or alternative medical intervention such as hospitalization. Described herein are methods, parenteral pharmaceutical formulations, and devices to meet these needs. Provided herein are methods, pharmaceutical formulations, and devices for the preventative treatment (prophylaxis) of incidental opioid overdose comprising the parenteral administration of a pharmaceutical formulation containing naloxone, naltrexone, or nalmefene as a prophylactic measure using an auto-injection device; i.e. “auto injector”.

Accordingly, in one aspect, the invention provides methods of preventing incidental opioid overdose or a symptom thereof. The method includes parenterally administering to a subject in need thereof a therapeutically effective amount of naloxone or a pharmaceutically acceptable salt thereof, wherein the therapeutically effective amount is equivalent to about 3.0 mg to about 10.0 mg of naloxone and/or a salt and/or solvate thereof, e.g., naloxone hydrochloride.

Accordingly, in another aspect, the invention provides methods of preventing incidental opioid overdose or a symptom thereof. The method includes parenterally administering to a subject in need thereof a therapeutically effective amount of naltrexone or a pharmaceutically acceptable salt thereof, wherein the therapeutically effective amount is equivalent to about 2.0 mg to about 8.0 mg of naltrexone and/or a salt and/or solvate thereof, e.g., naltrexone hydrochloride.

Accordingly, in yet another aspect, the invention provides methods of preventing incidental opioid overdose or a symptom thereof. The method includes parenterally administering to a subject in need thereof a therapeutically effective amount of nalmefene or a pharmaceutically acceptable salt thereof, wherein the therapeutically effective amount is equivalent to about 0.5 mg to about 2.0 mg of nalmefene and/or a salt and/or solvate thereof, e.g., nalmefene hydrochloride.

Also provided are devices adapted for parenteral delivery of a pharmaceutical formulation to a subject, comprising one or more doses of a therapeutically effective amount of nalmefene or a pharmaceutically acceptable salt thereof, wherein the device can be self-administered with little or no prior training, and wherein the therapeutically effective amount per dose is equivalent to about 3.0 mg to about 10.0 mg of naloxone or to about 2.0 to about 8.0 of naltrexone or about 0.5 mg to about 2.0 mg of nalmefene and/or a salt and/or solvate thereof; e.g. nalmefene hydrochloride.

In some embodiments, the parenteral pharmaceutical formulation is self-administered prior to a drug raid, e.g. by law enforcement personnel if incidental exposure to an opioid agonist such as fentanyl is anticipated.

In some embodiments, the parenteral pharmaceutical formulation is self-administered by warfighters if exposure to an opioid agonist such as fentanyl is anticipated.

In some embodiments, the formulation comprises a sterile aqueous solution.

In some embodiments, the formulation is equivalent to about 3.0 mg to about 10.0 mg per dose of naloxone and/or a salt and/or solvate thereof, e.g., naloxone hydrochloride.

In some embodiments, the formulation is equivalent to about 2.0 mg to about 8.0 mg per dose of naltrexone and/or a salt and/or solvate thereof, e.g., naltrexone hydrochloride.

In some embodiments, the formulation is equivalent to about 0.5 mg to about 2 mg per dose of nalmefene and/or a salt and/or solvate thereof, e.g., nalmefene hydrochloride.

In some embodiments, about 300 μL-1.0 mL mL of said formulation is delivered to the subject.

In some embodiments, the pharmaceutical formulation comprising a therapeutically effective amount of naloxone is administered in conjunction with an excipient. In some embodiments, the pharmaceutical formulation additionally comprises one or more excipients selected from sodium chloride, benzalkonium chloride, edetate disodium, and an acid such as hydrochloric acid. In some embodiments, the acid is sufficient to achieve a pH of about 3.9.

In some embodiments, the pharmaceutical formulation comprising a therapeutically effective amount of naltrexone is administered in conjunction with an excipient. In some embodiments, the pharmaceutical formulation additionally comprises one or more excipients selected from sodium chloride, benzalkonium chloride, edetate disodium, and an acid such as hydrochloric acid. In some embodiments, the acid is sufficient to achieve a pH of about 3.9.

In some embodiments, the pharmaceutical formulation comprising a therapeutically effective amount of nalmefene is administered in conjunction with an excipient. In some embodiments, the pharmaceutical formulation additionally comprises one or more excipients selected from sodium chloride, benzalkonium chloride, edetate disodium, and an acid such as hydrochloric acid. In some embodiments, the acid is sufficient to achieve a pH of about 3.9.

In some embodiments, the therapeutically effective amount comprises about 3 mg to about 10 mg, about 4.5 mg to about 8.5 mg, or about 6.0 mg to about 7.0 mg of naloxone. In some embodiments, the therapeutically effective amount comprises about 3.0 mg, about 4.0 mg, about 5.0 mg, about 6.0 mg, about 7.0 mg, about 8.0 mg, about 9.0 mg, or about 10.0 mg of naloxone.

In some embodiments, the therapeutically effective amount of naloxone is administered in doses of 3.0 mg to about 10.0 mg prior to, contemporaneously, or after incidental exposure to an opioid agonist.

Disclosed herein is a method of achieving a plasma concentration of naloxone therapeutically effective to treat incidental exposure to an opioid agonist in a subject in need thereof. The method comprises the parenteral administration of a pharmaceutical formulation comprising between about 3.0 mg and about 10.0 mg naloxone or a salt or hydrate thereof.

Also disclosed herein is a sterile, parenteral pharmaceutical formulation comprising naloxone and other excipients that achieves plasma concentrations with a Cₘₐₓ of ≥1 ng/ml within 15 minutes after intramuscular injection.

In some embodiments, the therapeutically effective amount comprises about 2.0 mg to about 8.0 mg, about 2.5 mg to about 5.5 mg, or about 3.0 to about 5.0 mg of naltrexone. In some embodiments, the therapeutically effective amount comprises about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg or about 8.0 mg of naltrexone.

In some embodiments, the therapeutically effective amount of naltrexone is administered in doses of 2.0 mg to about 8.0 mg prior to, contemporaneously, or after incidental exposure to an opioid agonist.

Disclosed herein is a method of achieving a plasma concentration of naltrexone therapeutically effective to treat incidental exposure to an opioid agonist in a subject in need thereof. The method comprises the parenteral administration of a pharmaceutical formulation comprising between about 2.0 mg and about 8.0 mg naltrexone or a salt or hydrate thereof.

Also disclosed herein is a sterile, parenteral pharmaceutical formulation comprising naltrexone and other excipients that achieves plasma concentrations with a Cₘₐₓ of ≥1 ng/ml within 15 minutes after intramuscular injection.

In some embodiments, the therapeutically effective amount comprises about 0.5 mg to about 2 mg, about 0.8 mg to about 1.7 mg, or about 1.1 to about 1.4 mg of nalmefene. In some embodiments, the therapeutically effective amount comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of nalmefene.

In some embodiments, the therapeutically effective amount of nalmefene is administered in doses of 0.5 mg to about 2 mg prior to, contemporaneously, or after incidental exposure to an opioid agonist.

Disclosed herein is a method of achieving a plasma concentration of nalmefene therapeutically effective to treat incidental exposure to an opioid agonist in a subject in need thereof. The method comprises the parenteral administration of a pharmaceutical formulation comprising between about 0.5 mg and about 2 mg nalmefene or a salt or hydrate thereof.

Also disclosed herein is a sterile, parenteral pharmaceutical formulation comprising nalmefene and other excipients that achieves plasma concentrations with a Cₘₐₓ of ≥1 ng/ml within 15 minutes after intramuscular injection.

### DETAILED DESCRIPTION

Provided herein are methods, parenteral pharmaceutical formulations, and devices for the preventative treatment (prophylaxis) of incidental opioid overdose comprising the self-administration of a sterile, parenteral pharmaceutical formulation containing the opioid antagonist naloxone, naltrexone, or nalmefene as a prophylactic measure.

Also disclosed herein are methods and pharmaceutical formulations for the prevention of opioid overdose and symptoms thereof, comprising administering a sterile, parenteral pharmaceutical formulation of naloxone, naltrexone, or nalmefene as a solution alone or in combination with other excipients.

Provided are devices adapted for parenteral (intramuscular or subcutaneous) self-delivery of a pharmaceutical formulation to a subject, comprising a therapeutically effective amount of the opioid antagonist naloxone, naltrexone, or nalmefene and pharmaceutically acceptable salts thereof, wherein the device is pre-primed, and wherein the therapeutically effective amount, is equivalent to about 3.0 mg to about 10.0 mg of naloxone or 2.0 mg to about 8.0 mg of naltrexone or 0.5 mg to about 2 mg of nalmefene and/or a salt and/or solvate thereof, e.g., nalmefene hydrochloride.

Also provided are methods of treating incidental exposure to an opioid agonist, comprising a subject self-administering by either intramuscular or subcutaneous injection, a therapeutically effective amount of the opioid antagonist naloxone, naltrexone, or nalmefene and pharmaceutically acceptable salts thereof, wherein the therapeutically effective amount is equivalent to about 3.0 mg to about 10.0 mg of naloxone or 2.0 mg to about 8.0 mg of naltrexone or 0.5 mg to about 2 mg of nalmefene and/or a salt and/or solvate thereof, e.g., nalmefene hydrochloride.

As used herein, the following terms have their meanings indicated.

Opioid receptors are G protein-coupled receptors (GPCRs) that are activated by endogenous opioid peptides, by clinically important alkaloid analgesic drugs such as morphine, and by synthetic, i.e., neither derived from nor present in opium poppies, analgesics such as methadone and fentanyl. There are three principal types of opioid receptors: the δ-opioid receptor, the κ-opioid receptor, and the μ-opioid receptor. Opioids depress respiration, which is controlled principally through medullary respiratory centers with peripheral input from chemoreceptors and other sources. Opioids produce inhibition at the chemoreceptors via mu opioid receptors and in the medulla via mu and possibly delta receptors. While multiple neurotransmitters participate in the control of respiration, glutamate and γ-aminobutyric acid (GABA) are the major excitatory and inhibitory neurotransmitters, respectively. This explains the potential for interaction of opioids with benzodiazepines and alcohol: both benzodiazepines and alcohol facilitate the inhibitory effect of GABA at GABAA receptors, while alcohol also decreases the excitatory effect of glutamate at NMDA receptors. Oxycodone and other opioid analgesics (such as hydrocodone and fentanyl) as well as heroin and methadone are all implicated in fatal overdose.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

The term “active ingredient” or “pharmaceutically active compound” is defined in the context of a “pharmaceutical formulation” and is intended to mean a component of a pharmaceutical formulation that provides the primary pharmacological effect, as opposed to an “inactive ingredient” which would generally be recognized as providing no pharmaceutical benefit.

The term “actuation,” as used herein, refers to operation of the device such that the pharmaceutical formulation is delivered therefrom.

The term “agonist,” as used herein, refers to a moiety that interacts with, and activates, a receptor and thereby initiates a physiological or pharmacological response characteristic of that receptor. The term “antagonist,” as used herein, refers to a moiety that interacts with or binds to a receptor at the same site as an agonist (for example, either an endogenous ligand or drug molecule) or at an allosteric site, but which does not activate the intracellular response initiated by the agonist and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “antimicrobial preservative,” as used herein, refers to a pharmaceutically acceptable excipient with antimicrobial properties which is added to a pharmaceutical composition to maintain microbiological stability.

The term “AUC,” as used herein, refers to the area under the drug plasma concentration-time curve. The term “AUC₀₋₄,” as used herein, refers to the area under the drug plasma concentration-time curve from t=0 to the last measurable concentration. The term “AUC_{0-∞},” as used herein, refers to the area under the drug plasma concentration-time curve extrapolated to ∞.

The term “bioavailability (F),” as used herein, refers to the fraction of a dose of drug that is absorbed from its site of administration and reaches, in an unchanged form, the systemic circulation. The term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term “relative bioavailability (Frei)” is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. The term “apparent clearance (CL/F),” as used herein, refers to clearance that does not take into account the bioavailability of the drug. It is the ratio of the dose over the AUC.

The term “Cₘₐₓ,” as used herein, refers to the maximum observed plasma concentration.

The term “coefficient of variation (CV),” as used herein, refers to the ratio of the sample standard deviation to the sample mean. It is often expressed as a percentage.

The term “device,” as used herein, refers to an apparatus capable of delivering a drug to subject in need thereof.

The term “delivery time,” as used herein, refers to the amount of time that elapses between a determination made by a healthcare professional, first responder, law enforcement (e.g., police, customs, and border patrol agents), military personnel, or an untrained individual, that s/he or another individual is in need of an opioid antagonist and completion of the delivery.

The term “disease,” as used herein, is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “drug raid,” as used herein, refers to the entry into and investigation of a facility in which opioids are manufactured, stored, and/or distributed. It typically connotes such a facility in violation of the laws regulating controlled substances.

The term “elimination rate constant (λ),” as used herein, refers to the fractional rate of drug removal from the body. This rate is constant in first-order kinetics and is independent of drug concentration in the body. λ is the slope of the plasma concentration-time line (on a logarithmic y scale). The term “λz,” as used herein, refers to the terminal phase elimination rate constant, wherein the “terminal phase” of the drug plasma concentration-time curve is a straight line when plotted on a semi-logarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

The term “equivalent,” as used herein, refers to a weight of an opioid antagonist selected from nalmefene and pharmaceutically acceptable salts thereof that is equimolar to a specified weight of nalmefene hydrochloride.

The term “excipient,” as used herein, refers to a natural or synthetic substance formulated alongside the active ingredient of a medication, included for long-term stabilization, bulking up solid formulations, or to confer a therapeutic enhancement on the active ingredient in the final dosage form, such as facilitating drug absorption, reducing viscosity, or enhancing solubility.

The term “filled,” as used herein, refers to an association between a device and a pharmaceutical composition, for example, when a pharmaceutical formulation described herein comprising a therapeutically effective amount of an opioid antagonist is present within a reservoir that forms a part of a device described herein.

The term “hydrate,” as used herein, refers to an opioid antagonist described herein or a salt thereof that further includes a stoichiometric or non-stoichiometric amount of water bound by non-covalent intermolecular forces.

An individual “who is at risk for incidental opioid overdose”, includes an individual who is accidentally exposed to opioids and/or anticipates incidental opioid exposure and may self-administer an intramuscular or subcutaneous injection using an auto-injection device.

As used herein, two embodiments are “mutually exclusive” when one is defined to be something which is different than the other. For example, an embodiment wherein the amount of nalmefene hydrochloride is specified to be 0.5 mg is mutually exclusive with an embodiment wherein the amount of nalmefene hydrochloride is specified to be 2 mg. However, an embodiment wherein the amount of nalmefene hydrochloride is specified to be 0.5 mg is not mutually exclusive with an embodiment in which the pharmaceutical formulation comprises an aqueous solution of about 300 μL-1.0 mL.

The term “naloxone,” as used herein, refers to a compound of the following structure:

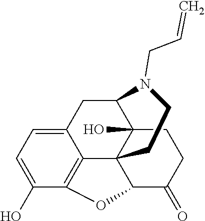

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (−)-17-allyl-4,5α-epoxy-3,14-dihydroxymorphinan-6-one; 4,5α-epoxy-3,14-dihydroxy-17-(2-propenyl)morphinan-6-one; and (−)-12-allyl-7,7a,8,9-tetrahydro-3,7a-dihydroxy-4aH-8,9c-iminoethanophenanthro[4,5-bcd]furan-5(6H)-one. Naloxone hydrochloride may be anhydrous (CAS Reg. No. 357-08-4) and also forms a dihydrate (CAS No. 51481-60-8). It has been sold under various brand names including Narcan®, Nalone®, Naloxone®, Naloxona®, Naloxonum®, Narcanti®, and Narcon®.

The term “naltrexone,” as used herein, refers to a compound of the following structure:

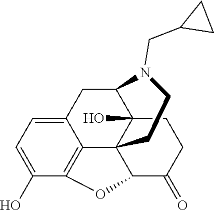

or a pharmaceutically acceptable salt, hydrate, or solvate thereof. The CAS registry number for naltrexone is 16590-41-3. Other names for naltrexone include: 17-(cyclopropylmethyl)-4,5α-epoxy-3,14-dihydroxymorphinan-6-one; (5a)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacyclo[9.6.1.01,13.05,17.07,18]octadeca-7(18),8,10-trien-14-one. Naltrexone hydrochloride (CAS Reg. No. 16676-29-2) has been marketed under the trade names Antaxone®, Depade®, Nalorex®, Revia®, Trexan®, Vivitrex®, and Vivitrol®.

The term “nalmefene,” as used herein, refers to 17-cyclopropylmethyl-4,5α-epoxy-6-methylenemorphinan-3,14-diol, a compound of the following structure:

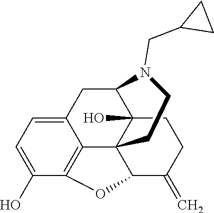

Nalmefene hydrochloride (CAS Reg. No. 58895-64-0) has been marketed under the trade names Nalmetrene®, Cervene®, Revex®, Arthrene®, and Incystene®.

Nalmefene, naltrexone or naloxone may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. The acid addition salts may be obtained as the direct products of compound synthesis. In the alternative, the free base may be dissolved in a suitable solvent containing the appropriate acid and the salt isolated by evaporating the solvent or otherwise separating the salt and solvent. Nalmefene and its salts may form solvates with standard low molecular weight solvents using methods known to the skilled artisan.

The term “opioid overdose,” as used herein, refers to an acute medical condition caused by incidental exposure to an opioid agonist in a subject. Symptoms of opioid overdose include including respiratory depression, central nervous system depression (which may include sedation, altered level consciousness, miotic (constricted) pupils), and cardiovascular depression (which may include hypoxemia and hypotension). Visible signs of opioid overdose or suspected opioid overdose include: unresponsiveness and/or loss of consciousness (unresponsive to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting.

The term “pharmaceutical formulation, or equivalently, “pharmaceutical composition,” as used herein, refers to a formulation comprising at least one active ingredient; including but not limited to nalmefene and/or salts, solvates and/or hydrates thereof, together with at least one pharmaceutically acceptable carrier or excipient, whereby the formulation is amenable to use for a specified, efficacious outcome in a subject (for example, without limitation, a human).

The term “pharmaceutically acceptable,” as used herein, refers to a component of a pharmaceutical formulation that is compatible with the other ingredients of the formulation and not overly deleterious to the recipient thereof.

As used herein, the term “protective packaging” refers to overwrap.

The term “receptor binding or occupancy” refers to a characterization of the kinetics between a radioactive drug and receptors or other binding sites throughout the body, and characterization of the radioactive drug binding affinity to these receptors.

The term “providing” in the context of providing a co-packaged drug product as disclosed herein to an individual includes co-packaging the drug product, prescribing the co-packaged drug product, and dispensing the co-packaged drug product. The providing may be done either directly to an individual (for example, to an individual for whom an opioid agonist prescription is appropriate, or who is otherwise at risk of opioid overdose) or to a second individual.

The term “solvate,” as used herein, refers to an opioid antagonist described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “sterile filling,” as used herein, refers methods of manufacturing the devices and pharmaceutical formulations described herein, such that the use of preservatives is not required. Sterile drug products may be produced using aseptic processing or terminal sterilization. Terminal sterilization usually involves filling and sealing product containers under high-quality environmental conditions. In an aseptic process, the drug product, container, and closure are first subjected to sterilization methods separately, as appropriate, and then brought together.

The term “storage-stable,” as used herein, refers to a pharmaceutical formulation in which at least about 90% to 99.5% of the active ingredient remains in an undegraded state after storage of the pharmaceutical formulation at specified temperature and humidity for a specified time, for example, for 12 months at 25° C. and 60% relative humidity.

The term “subject” as used herein refers to any living individual (preferably human) likely to benefit from treatment with a therapeutically effective amount of nalmefene. In some embodiments, the subject is exposed incidentally to an opioid agonist, such as fentanyl. In additional embodiments, the subject may include, but is not limited to, members of the military, law enforcement, professional security personnel, or personnel providing emergency medical services.

The term “substantially free of antimicrobial preservatives” is understood by one of ordinary skill in the art to described a pharmaceutical formulation that comprises less than 1% w/w antimicrobial preservatives.

“Prophylaxis” as used herein is synonymous with the terms “preventative treatment” and “prevention,” and is to be considered in its broadest context and refers to any medical or public health procedure employed to prevent a disease or condition, such as opioid overdose or a symptom thereof, from occurring. It does not necessarily mean that the subject will not eventually contract a disease or condition or experience a symptom. For example, if a long-acting opioid agonist is encountered by a person and enters the person's body, a short-acting opioid antagonist may prevent opioid intoxication or overdose for as long as it is active; the fact that opioid intoxication or overdose may later occur when the antagonist wears off does not mean that the antagonist does not “prevent.” Accordingly, prophylaxis may include the mitigation or amelioration of the symptoms of a disease or condition or preventing or reducing the risk of developing a disease or condition or symptoms thereof. The term “prophylaxis” may include reducing the severity of the onset of a disease or condition.

“Therapeutically effective amount” or “therapeutically effective dose”, as used herein means effective to prevent opioid overdose or symptoms thereof caused by incidental exposure of an opioid agonist in a subject. Opioid overdose may be moderate, severe, or even fatal. In some embodiments, a treatment or pharmaceutical formulation will be therapeutically effective to prevent even moderate opioid overdose, wherein a subject is temporarily physically or mentally impaired by an opioid agonist but is in no danger of impairment or harm beyond the agonist's excretion from the subject. In some embodiments, a treatment or formulation will be therapeutically effective to prevent severe overdose, wherein a subject is in danger of lasting or even permanent harm. In some embodiments, a treatment or formulation will be therapeutically effective to prevent fatal overdose.

“Exposure” as used herein refers to an actual or anticipated contact between the subject and an opioid agonist. Actual exposure refers to exposure that in fact occurs whether known or unknown. Anticipated exposure refers to any level of expected possibility of being exposed to an opioid.

“Incidental exposure to an opioid agonist by a subject” as used herein means that the exposure to an opioid agonist is not voluntary and/or intended to self-intoxicate, but occurs as part of the subject's activities in proximity to, or potential proximity to, the opioid agonist. For example, workers acting in capacity as law enforcement, inspectors, public health field agents, may face the risk of incidental exposure during activities such as cleaning or performing inspections or investigations for and/or handling opioid agonists or materials that are associated with opioid agonists, such as laboratory equipment, containers, needles, pipes or other objects. Additionally, military, law enforcement, or security personnel may face incidental exposure to opioid agonist when a third party deliberately delivers an opioid agonist to incapacitate said personnel, for example as a gas. Accordingly, incidental exposure to opioid agonist includes, for example, exposure by inhalation to aerosolized opioid agonist and incidental transdermal exposure to opioid agonist.

“Aerosolized opioid agonist” as used herein means that the opioid agonist is delivered through the air to a subject as, e.g., a gas, mist, or fine powder. It does not include opioid agonist in powder form that is voluntarily inhaled (e.g., snorted) by a subject. An example of an aerosolized opioid agonist is fentanyl (or a derivative thereof) administered through the ventilation system of a building to anaesthetize, incapacitate, or kill the occupants.

The term “t_{1/2}” or “half-life,” as used herein, refers to the amount of time required for half of a drug (for example, an opioid or an opioid antagonist) to be eliminated from the body or the time required for a drug concentration to decline by half.

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

The term “tomography,” as used herein, refers to a process of imaging by sections. The images may be looked at individually, as a series of two-dimensional slices or together, as a computer-generated three-dimensional representation.

The term “Tₘₐₓ,” as used herein, refers to the time from administration of the pharmaceutical formulations described herein to maximum drug plasma concentration.

The term “untrained individual” refers to an individual administering to him/herself or another subject an opioid antagonist using a device described herein, wherein the individual is not a healthcare professional and has received either no or minimal training in the use of the device.

### Opioid Antagonists

Provided are drug products adapted for parenteral delivery of an opioid receptor antagonist using an auto-injector device. Opioid receptor antagonists are a well-recognized class of chemical agents. They have been described in detail in the scientific and patent literature. Opioid antagonists, such as naloxone, naltrexone, and nalmefene, are agents which specifically reverse the effects of opioid agonists but have no opioid agonist activity.

### Naloxone

Naloxone is commercially available as a hydrochloride salt and has been sold under various brand names including Narcan®, Nalone®, Nalossone®, Naloxona®, Naloxonum®, Narcanti®, and Narcon®, and has been approved for opioid overdose reversal, and as disclosed herein, can be used to prevent incidental exposure to an opioid agonist that can be quickly and easily administered via an auto-injector device as a prophylactic measure by first responders, law enforcement (e.g., police, customs, and border patrol agents) and military personnel if contact with opioids, especially high potency synthetics, is either anticipated or suspected.

Provided are pharmaceutical formulations, devices adapted for parenteral delivery of an opioid receptor antagonist using an auto-injector device to a subject, kits comprising the foregoing, and methods of using the same in the prevention (prophylaxis) of opioid overdose, or symptoms thereof, each comprising a therapeutically effective amount of an opioid antagonist. In some embodiments, the opioid antagonist is selected from naloxone and/or pharmaceutically acceptable salts and/or hydrates thereof. In some embodiments, the therapeutically effective amount of naloxone and/or pharmaceutically acceptable salts and/or hydrates thereof is equivalent to about 3.0 mg to about 10.0 mg.

In some embodiments, naloxone is the only pharmaceutically active compound in pharmaceutical formulation. In some embodiments, naloxone is naloxone hydrochloride. In some embodiments, naloxone is anhydrous naloxone hydrochloride.

In some embodiments, the therapeutically effective amount of naloxone is between about 3 mg to about 10 mg, about 4.5 mg to about 8.5 mg, or about 6.0 mg to about 7.0 mg of naloxone. In some embodiments, the therapeutically effective amount comprises about 3.0 mg, about 4.0 mg, about 5.0 mg, about 6.0 mg, about 7.0 mg, about 8.0 mg, about 9.0 mg, or about 10.0 mg of naloxone.

In some embodiments, the nalmefene or other opioid antagonist is provided in a pharmaceutical formulation for parenteral administration comprising an aqueous solution of not more than about 300 μL-1.0 mL. In some embodiments, the parenteral pharmaceutical formulation comprises between about 3.0 mg to about 10.0 mg of naloxone; and between about 2.7 mg and about 4.5 mg of an isotonicity agent.

In some embodiments, the nalmefene is provided in a parenteral pharmaceutical formulation comprising an aqueous solution of not more than about 300 μL-1.0 mL. In some embodiments, the naloxone is provided at a concentration of between about 0.1% (w/v) and about 0.67% (w/v).

In some embodiments, the naloxone is provided in a parenteral pharmaceutical formulation comprising an aqueous solution which also comprises at least one additional excipient. In some embodiments, one such excipient is an isotonicity agent. In some embodiments, parenteral pharmaceutical formulation is an aqueous solution of not more than about 300 μL-1.0 mL comprising between about 0.1% (w/v) and about 0.67% (w/v) of nalmefene and between about 0.2% (w/v) and about 1.2% (w/v) of an isotonicity agent.

In some embodiments, the naloxone is provided in a parenteral pharmaceutical formulation comprising an aqueous solution comprising between about 3.0 mg to about 10.0 mg naloxone and/or an equivalent amount of a salt and/or solvate thereof, e.g., naloxone hydrochloride; and between about 2.7 mg and about 4.5 mg of an isotonicity agent.

In some embodiments, the isotonicity agent is sodium chloride (NaCl).

In some embodiments, the pharmaceutical formulation further comprises one or more excipients selected from water, NaCl, and hydrochloric acid. In some embodiments, the pharmaceutical formulation further comprises water, NaCl, and hydrochloric acid.

Also provided herein is a method for the prevention (prophylaxis) of opioid overdose or a symptom thereof caused by incidental exposure of a subject to an opioid agonist, comprising self-administering an intramuscular or subcutaneous injection using an auto-injection device, to a subject, in need thereof, a pharmaceutical formulation comprising:
about 3.0 mg to about 10.0 mg naloxone and/or an equivalent amount of a salt and/or solvate thereof; e.g. naloxone hydrochloride;
between about 0.1 to about 6.0 mg of an isotonicity agent;
optionally a stabilizing agent; and
an amount of acid or base sufficient to achieve a pH of 3.4-4.4.

In some embodiments, the pharmaceutical formulation comprises: about 3.0 mg to about 10.0 mg naloxone and/or an equivalent amount of a salt and/or solvate thereof; e.g., naloxone hydrochloride; about 2.7 mg to about 4.5 mg of sodium chloride; and an amount of hydrochloric acid sufficient to achieve a pH of about 3.9.

In some embodiments, the pharmaceutical formulation comprising about 3.0 mg to about 10.0 mg naloxone disclosed herein is sterilized via methods by any means known and available to one skilled in the art. Sterilization methods are discussed below.

### Naltrexone

Naltrexone is commercially available as a hydrochloride salt and has been marketed under the trade names Antaxone®, Depade®, Nalorex®, Revia®, Trexan®, Vivitrex®, and Vivitrol®.

Provided are pharmaceutical formulations, devices adapted for parenteral delivery of an opioid receptor antagonist using an auto-injector device to a subject, kits comprising the foregoing, and methods of using the same in the prevention (prophylaxis) of opioid overdose, or symptoms thereof, each comprising a therapeutically effective amount of an opioid antagonist. In some embodiments, the opioid antagonist is selected from naltrexone and/or pharmaceutically acceptable salts and/or hydrates thereof. In some embodiments, the therapeutically effective amount of naltrexone hydrochloride and/or pharmaceutically acceptable salts and/or hydrates thereof is equivalent to about 2.0 mg to about 8.0 mg.

In some embodiments, naltrexone is the only pharmaceutically active compound in pharmaceutical formulation. In some embodiments, naltrexone is naltrexone hydrochloride. In some embodiments, naltrexone is anhydrous naltrexone hydrochloride.

In some embodiments, the therapeutically effective amount of naltrexone is 2.0 mg to about 6.0 mg, about 2.5 mg to about 5.5 mg, or about 3.0 to about 5.0 mg of naltrexone. In some embodiments, the therapeutically effective amount comprises about 2.0 mg, about 2.5 mg, about 3.0 mg, about 3.5 mg, about 4.0 mg, about 4.5 mg, about 5.0 mg, about 5.5 mg, about 6.5 mg, about 7.0 mg, about 7.5 mg or about 8.0 mg of naltrexone.

In some embodiments, the naltrexone or other opioid antagonist is provided in a pharmaceutical formulation for parenteral administration comprising an aqueous solution of not more than about 300 μL-1.0 mL. In some embodiments, the parenteral pharmaceutical formulation comprises between about 3.0 mg to about 10.0 mg of naltrexone; and between about 2.7 mg and about 4.5 mg of an isotonicity agent.

In some embodiments, the naltrexone is provided in a parenteral pharmaceutical formulation comprising an aqueous solution of not more than about 300 μL-1.0 mL. In some embodiments, the naltrexone is provided at a concentration of between about 0.1% (w/v) and about 0.67% (w/v).

In some embodiments, the naltrexone is provided in a parenteral pharmaceutical formulation comprising an aqueous solution which also comprises at least one additional excipient. In some embodiments, one such excipient is an isotonicity agent. In some embodiments, parenteral pharmaceutical formulation is an aqueous solution of not more than about 300 μL-1.0 mL comprising between about 0.1% (w/v) and about 0.67% (w/v) of nalmefene and between about 0.2% (w/v) and about 1.2% (w/v) of an isotonicity agent.

In some embodiments, the naltrexone is provided in a parenteral pharmaceutical formulation comprising an aqueous solution comprising between about 2.0 mg to about 8.0 mg naltrexone and/or an equivalent amount of a salt and/or solvate thereof, e.g., naltrexone hydrochloride; and between about 2.7 mg and about 4.5 mg of an isotonicity agent.

In some embodiments, the isotonicity agent is sodium chloride (NaCl).

In some embodiments, the pharmaceutical formulation further comprises one or more excipients selected from water, NaCl, and hydrochloric acid. In some embodiments, the pharmaceutical formulation further comprises water, NaCl, and hydrochloric acid.

Also provided herein is a method for the prevention (prophylaxis) of opioid overdose or a symptom thereof caused by incidental exposure of a subject to an opioid agonist, comprising self-administering an intramuscular or subcutaneous injection using an auto-injection device, to a subject, in need thereof, a pharmaceutical formulation comprising:
about 2.0 mg to about 8.0 mg naltrexone and/or an equivalent amount of a salt and/or solvate thereof; e.g. naltrexone hydrochloride;
between about 0.1 to about 6.0 mg of an isotonicity agent;
optionally a stabilizing agent; and
an amount of acid or base sufficient to achieve a pH of 3.4-4.4.

In some embodiments, the pharmaceutical formulation comprises: about 2.0 mg to about 8.0 mg naltrexone and/or an equivalent amount of a salt and/or solvate thereof; e.g., naltrexone hydrochloride; about 2.7 mg to about 4.5 mg of sodium chloride; and an amount of hydrochloric acid sufficient to achieve a pH of about 3.9.

In some embodiments, the pharmaceutical formulation comprising about 2.0 mg to about 8.0 mg naltrexone disclosed herein is sterilized via methods by any means known and available to one skilled in the art. Sterilization methods are discussed below.

### Nalmefene

Nalmefene is commercially available as a hydrochloride salt and is a 6-methylene analog of naltrexone. Nalmefene hydrochloride (17-(cyclopropylmethyl)-4,5(-epoxy-6-methylenemorphinan-3,14-diol) has been approved for opioid overdose reversal, and as disclosed herein, can be used to prevent incidental exposure to an opioid agonist that can be quickly and easily administered via an auto-injector device as a prophylactic measure by first responders, law enforcement (e.g., police, customs, and border patrol agents) and military personnel if contact with opioids, especially high potency synthetics, is either anticipated or suspected.

In individuals who are not opioid dependent (and therefore not at risk of a precipitated withdrawal such as could be produced in individuals who are physically dependent on opioids), very high intravenous doses of nalmefene (up to 24 mg) are safe and well tolerated, Dixon, R, Howes, J, Gentile, J. 1986. Clin. Pharmacol. Ther. 39:49-53. Further, nalmefene tablets (20 mg) have been approved in the European Union for treatment of alcohol use disorders, and it has been chronically administered to thousands of individuals. This underscores the potential safety profile of a nalmefene product administered as a prophylactic measure by individuals who are not opioid dependent on an as-needed basis when incidental contact with opioids is anticipated or expected.

Provided are pharmaceutical formulations, devices adapted for parenteral delivery of an opioid receptor antagonist using an auto-injector device to a subject, kits comprising the foregoing, and methods of using the same in the prevention (prophylaxis) of opioid overdose, or symptoms thereof, each comprising a therapeutically effective amount of an opioid antagonist. In some embodiments, the opioid antagonist is selected from nalmefene and/or pharmaceutically acceptable salts and/or hydrates thereof. In some embodiments, the therapeutically effective amount of nalmefene hydrochloride and/or pharmaceutically acceptable salts and/or hydrates thereof is equivalent to about 0.5 mg to about 2.0 mg.

In some embodiments, nalmefene is the only pharmaceutically active compound in pharmaceutical formulation. In some embodiments, nalmefene is nalmefene hydrochloride. In some embodiments, nalmefene is anhydrous nalmefene hydrochloride.

In some embodiments, the therapeutically effective amount of nalmefene is between about 0.5 mg to about 2.0 mg, about 0.8 mg to about 1.7 mg, about 1.1 to about 1.4 of nalmefene. In some embodiments, the therapeutically effective amount comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of nalmefene.

In some embodiments, the nalmefene or other opioid antagonist is provided in a pharmaceutical formulation for parenteral administration comprising an aqueous solution of not more than about 300 μL-1.0 mL. In some embodiments, the parenteral pharmaceutical formulation comprises between about 0.5 mg and about 2.0 mg of nalmefene; and between about 2.7 mg and about 4.5 mg of an isotonicity agent.

In some embodiments, the nalmefene is provided in a parenteral pharmaceutical formulation comprising an aqueous solution of not more than about 300 μL-1.0 mL. In some embodiments, the nalmefene is provided at a concentration of between about 0.1% (w/v) and about 0.67% (w/v).

In some embodiments, the nalmefene is provided in a parenteral pharmaceutical formulation comprising an aqueous solution which also comprises at least one additional excipient. In some embodiments, one such excipient is an isotonicity agent. In some embodiments, parenteral pharmaceutical formulation is an aqueous solution of not more than about 300 μL-1.0 mL comprising between about 0.1% (w/v) and about 0.67% (w/v) of nalmefene and between about 0.2% (w/v) and about 1.2% (w/v) of an isotonicity agent.

In some embodiments, the nalmefene is provided in a parenteral pharmaceutical formulation comprising an aqueous solution comprising between about 0.5 mg and about 2.0 mg nalmefene and/or an equivalent amount of a salt and/or solvate thereof, e.g., nalmefene hydrochloride; and between about 2.7 mg and about 4.5 mg of an isotonicity agent.

In some embodiments, the isotonicity agent is sodium chloride (NaCl).

In some embodiments, the pharmaceutical formulation further comprises one or more excipients selected from water, NaCl, and hydrochloric acid. In some embodiments, the pharmaceutical formulation further comprises water, NaCl, and hydrochloric acid.

Also provided herein is a method for the prevention (prophylaxis) of opioid overdose or a symptom thereof caused by incidental exposure of a subject to an opioid agonist, comprising self-administering an intramuscular or subcutaneous injection using an auto-injection device, to a subject, in need thereof, a pharmaceutical formulation comprising:
about 0.5 mg to about 2.0 mg nalmefene and/or an equivalent amount of a salt and/or solvate thereof; e.g. nalmefene hydrochloride;
between about 0.1 to about 6.0 mg of an isotonicity agent;
optionally a stabilizing agent; and
an amount of acid or base sufficient to achieve a pH of 3.4-4.4.

In some embodiments, the pharmaceutical formulation comprises: about 0.5 mg to about 2.0 mg nalmefene and/or an equivalent amount of a salt and/or solvate thereof; e.g., nalmefene hydrochloride; about 2.7 mg to about 4.5 mg of sodium chloride; and an amount of hydrochloric acid sufficient to achieve a pH of about 3.9.

In some embodiments, the pharmaceutical formulation comprising about 0.5 mg to about 2.0 mg nalmefene disclosed herein is sterilized via methods by any means known and available to one skilled in the art. Sterilization methods are discussed below.

### Pharmaceutical Formulations

Provided herein are parenteral pharmaceutical formulations comprising one or more opioid antagonists. In some embodiments, the pharmaceutical formulations comprise an opioid antagonist and a pharmaceutically acceptable carrier. The carrier(s) must be “acceptable” in the sense of being compatible with the other ingredients of the formulation and not overly deleterious to the recipient thereof. Some embodiments of the present disclosure include a method of producing a pharmaceutical formulation comprising admixing at least one opioid antagonist and a pharmaceutically acceptable carrier.

Liquid preparations include solutions, suspensions and emulsions, for example, water or water-propylene glycol solutions. Additional ingredients in liquid preparations may include: antimicrobial preservatives, such as benzalkonium chloride (which may also act as a cationic surfactant and/or a absorption enhancer), methylparaben, sodium benzoate, benzoic acid, phenyl ethyl alcohol, and the like, and mixtures thereof surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof and a suspending agent such as microcrystalline cellulose, carboxymethylcellulose sodium NF, polyacrylic acid, magnesium aluminum silicate, xanthan gum, and the like, and mixtures thereof.

In some embodiments, provided herein are pharmaceutical formulations for parenteral administration comprising naloxone, naltrexone or nalmefene. Techniques well known to those in the art can be used to make a pharmaceutical formulation comprising nalmefene.

In some embodiments, nalmefene is the only pharmaceutically active compound in said pharmaceutical formulation.

In some embodiments, naloxone is naloxone hydrochloride, or a hydrate thereof. In some embodiments, the naloxone is naloxone hydrochloride.

In some embodiments, naltrexone is naltrexone hydrochloride or a hydrate thereof. In some embodiments, the naltrexone is naltrexone hydrochloride.

In some embodiments, nalmefene is nalmefene hydrochloride, or a hydrate thereof. In some embodiments, the nalmefene is nalmefene hydrochloride.

In some embodiments, the formulation is an aqueous solution. In some embodiments, the formulation comprises, per dose, between about 300 μL to about 1.0 mL of the aqueous solution.

In some embodiments, the parenteral pharmaceutical formulation comprises between about 0.1% (w/v) and about 0.67% (w/v). In some embodiments, the formulation comprises about 0.1% (w/v), about 0.2% (w/v), about 0.3% (w/v), about 0.4% (w/v), about 0.5% (w/v), about 0.6% or about 0.7% (w/v).

The pharmaceutical formulation may comprise any of the amounts of naloxone hydrochloride as provided above, for example, equivalent to about 3 mg to about 10 mg, about 4.5 mg to about 8.5 mg, or about 6.0 mg to about 7.0 mg of naloxone. In some embodiments, the therapeutically effective amount comprises about 3.0 mg, about 4.0 mg, about 5.0 mg, about 6.0 mg, about 7.0 mg, about 8.0 mg, about 9.0 mg, or about 10.0 mg of naloxone.

The pharmaceutical formulation may comprise any of the amounts of naltrexone hydrochloride as provided above, for example, equivalent to about 2.0 mg to about 8.0 mg, about 2.5 mg to about 5.5 mg, or about 3.0 to about 5.0 mg of naltrexone. In some embodiments, the therapeutically effective amount comprises about 2.0 mg, about 2.5 mg, about 3.0 mg, about 3.5 mg, about 4.0 mg, about 4.5 mg, about 5.0 mg, about 5.5 mg, about 6.5 mg, about 7.0 mg, about 7.5 mg or about 8.0 mg of naltrexone.

The pharmaceutical formulation may comprise any of the amounts of nalmefene hydrochloride as provided above, for example, equivalent to about 0.5 mg to about 2.0 mg. In some embodiments, the pharmaceutical formulation comprises about 0.5 mg to about 2 mg, about 0.8 mg to about 1.7 mg, about 1.1 to about 1.4 of nalmefene. In some embodiments, the therapeutically effective amount comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg of nalmefene.

In some embodiments, the pharmaceutical formulation comprises a solution prepared from naloxone hydrochloride.

In some embodiments, the pharmaceutical formulation comprises a solution prepared from naltrexone hydrochloride.

In some embodiments, the pharmaceutical formulation comprises a solution prepared from nalmefene hydrochloride.

In some embodiments, the pharmaceutical formulation further comprises one or more excipients selected from water and NaCl.

In some embodiments, the pharmaceutical formulation is substantially free of antimicrobial preservatives.

In some embodiments, the device is substantially free of benzalkonium chloride, methylparaben, sodium benzoate, benzoic acid, phenyl ethyl alcohol.

In some embodiments, the pharmaceutical formulation is storage-stable for about twelve months at about 25° C.

In some embodiments, the pharmaceutical formulation comprises less than 0.1% w/w antimicrobial preservatives. In some embodiments, the pharmaceutical formulation comprises 0.01% w/w or less antimicrobial preservatives. In some embodiments, the pharmaceutical formulation comprises 0.01% w/w-0.001% w/w antimicrobial preservatives. In some embodiments, the pharmaceutical formulation comprises less than 0.001% w/w antimicrobial preservatives.

In some embodiments, acid and/or base is sued to achieve a specific pH suitable for parenteral delivery, e.g. as an intramuscular injection. In some embodiments the acid or base, is sufficient to achieve a pH of about 3.4-4.4. In some embodiments the acid or base, is sufficient to achieve a pH of about 3.7-4.1. In some embodiments the acid or base, is sufficient to achieve a pH of about 3.7, about 3.8, about 3.9, about 4.0, or about 4.1.

Accordingly, provided herein is a parenteral pharmaceutical formulation in an aqueous solution of about 3004 μL-1.0 mL comprising:
a therapeutically effective amount of naloxone hydrochloride, naltrexone hydrochloride, or nalmefene hydrochloride or a hydrate thereof;
an isotonicity agent; and
an amount of acid or base sufficient to achieve a pH of 3.4-4.4

In some embodiments, the aqueous solution comprises:
between about 0.5 mg and about 2.0 mg of nalmefene hydrochloride;
between about 2.7 mg and about 4.5 mg of an isotonicity agent; and
   an amount of acid or base sufficient to achieve a pH of 3.4-4.4.

Accordingly, provided herein is a parenteral pharmaceutical formulation in an aqueous solution of about 300 μL-1.0 mL comprising:
a therapeutically effective amount of naltrexone hydrochloride or a hydrate thereof;
an isotonicity agent; and
an amount of acid or base sufficient to achieve a pH of 3.4-4.4

In some embodiments, the aqueous solution comprises:
between about 0.5 mg and about 2.0 mg of naltrexone hydrochloride;
between about 2.7 mg and about 4.5 mg of an isotonicity agent; and
an amount of acid or base sufficient to achieve a pH of 3.4-4.4.
   Accordingly, provided herein is a parenteral pharmaceutical formulation in an aqueous solution of about 300 μL-1.0 mL comprising:
a therapeutically effective amount of nalmefene hydrochloride or a hydrate thereof;
an isotonicity agent; and
an amount of acid or base sufficient to achieve a pH of 3.4-4.4
   In some embodiments, the aqueous solution comprises:
between about 0.5 mg and about 2.0 mg of nalmefene hydrochloride;
between about 2.7 mg and about 4.5 mg of an isotonicity agent; and
an amount of acid or base sufficient to achieve a pH of 3.4-4.4.

In some embodiments,
the isotonicity agent is NaCl; and
the acid is hydrochloric acid.

In some embodiments, the pharmaceutical formulation is storage-stable for about twelve months at about 25° C. and about 60% relative humidity.

Also provided are embodiments wherein any embodiment above may be combined with any one or more of these embodiments, provided the combination is not mutually exclusive.

### Auto-Injection Drug Delivery Devices and Kits

Also provided are drug delivery devices comprising a pharmaceutical formulation described herein. Disclosed herein are pharmaceutical formulations in a device adapted for delivery using an auto-injector device to a subject for prevention (prophylaxis) of opioid overdose or a symptom thereof, caused by incidental exposure of a subject to an opioid agonist. In some embodiments, the device can be actuated with one hand. The subject can actuate the auto injector and self-administer the formulation, or another person can actuate it. The actuating individual need not be a medically-trained individual. The auto-injector is adapted to be administered in the thigh (esp. the anterolateral aspect of the thigh), through clothing if necessary; however, it will be suitable for injection in other muscles, including the deltoid and gluteus maximus.

Parenteral delivery using an auto-injector is considered an attractive, safe, and easy-to-administer means of insuring systemic drug delivery, especially when rapid absorption and effect are desired. Auto-injector devices allow the individual to self administer an opioid antagonist as a prophylactic measure when incidental/occupational exposure to opioid agonists is anticipated or suspected. These device enable both self-administration and an individual to administer an injection to another individual with little or no training.

### Auto-Injectors

Auto-injectors are constructed in a manner which keeps the needle tip shielded prior to injection and also has a passive safety mechanism to prevent accidental firing (injection). Injection depth can be adjustable or fixed and a function for needle shield removal may be incorporated. Just by pressing a button, the syringe needle is automatically inserted and the drug is delivered. An example of this is the Evzio® (naloxone HCl injection) 2.0 mg auto-injector. Once the injection is completed some auto injectors have visual or audio indication to confirm that the full dose has been delivered. Some autoinjectors contain glass syringes, which can make them fragile and vulnerable to contamination. More recently, companies have been looking into making autoinjector syringes out of plastic to prevent this issue.

Some auto-injectors have the shape and size of a smartphone which can be put into a pocket. This design also has retractable needle and automated voice instructions to assist the users on how to correctly use the auto-injectors. The “Auvi-Q” epinephrine auto-injector is an example of this design.

In the military, auto-injectors are used to protect personnel from chemical warfare agents. In the U.S. military, atropine and 2-PAM-Cl (pralidoxime chloride) are used for first aid (“buddy aid” or “self aid”) against nerve agents. An issue item, the Mark I NAAK (Nerve Agent Antidote Kit), provides these drugs in the form of two separate autoinjectors. A newer model, the ATNAA (Antidote Treatment Nerve Agent Auto-Injector), has both drugs in one syringe, allowing for the simplification of administration procedures.

A newer variant of the auto-injector is the gas jet autoinjector, which contains a cylinder of pressurised gas and propels a fine jet of liquid through the skin without the use of a needle. Patients who fear needles may be more accepting of using these devices. The auto-injector can be reloaded, and a variety of different doses or different drugs can be used, although the only widespread application to date has been for the administration of insulin in the treatment of diabetes.

Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

Devices recited herein may employ any of the pharmaceutical formulations, and are useful in all the methods, disclosed herein.

In some embodiments, the subject using the device is an opioid overdose subject caused by incidental exposure of the subject to an opioid agonist.

In some embodiments, the device is actuated and the opioid antagonist, e.g., naloxone, naltrexone, or nalmefene, is delivered, by an untrained individual, which in some cases, may be the subject anticipating incidental exposure to an opioid agonist, and on other cases, may be another individual.

Accordingly, provided herein is a drug product comprising, in a single-use auto-injector device adapted for parenteral delivery of a pharmaceutical formulation to a subject by self-administration, a pharmaceutical formulation which is an aqueous solution of about 0.5-1.0 mL comprising:
naloxone hydrochloride or a hydrate thereof;
an isotonicity agent; and
an amount of acid or base sufficient to achieve a pH of 3.4-4.4

In some embodiments, the drug product comprises a single-use auto-injector device adapted for parenteral delivery of a pharmaceutical formulation to a subject by self-administration, a pharmaceutical formulation which is an aqueous solution of about 300 μL-1 mL comprising:
naltrexone hydrochloride or a hydrate thereof;
an isotonicity agent; and
an amount of acid or base sufficient to achieve a pH of 3.4-4.4

In some embodiments, the drug product comprises a single-use auto-injector device adapted for parenteral delivery of a pharmaceutical formulation to a subject by self-administration, a pharmaceutical formulation which is an aqueous solution of about 300-500 μL comprising:
nalmefene hydrochloride or a hydrate thereof;
an isotonicity agent; and
an amount of acid or base sufficient to achieve a pH of 3.4-4.4

In some embodiments, the single-use auto injector device comprises any of the amounts of nalmefene hydrochloride provided above; e.g. between about 3.0-10.0 mg of the nalmefene hydrochloride or a hydrate thereof.

In some embodiments, the single-use auto injector device comprises any of the amounts of naltrexone hydrochloride provided above; e.g. between about 2.0-8.0 mg of the naltrexone hydrochloride or a hydrate thereof.

In some embodiments, the single-use auto injector device comprises any of the amounts of nalmefene hydrochloride provided above; e.g. between about 0.5 to about 2.0 mg of the nalmefene hydrochloride or a hydrate thereof.

In some embodiments, the single-use auto injector device comprises any of the parenteral pharmaceutical formulations disclosed above.

In some embodiments, the device is a single-dose device, wherein the pharmaceutical formulation is present in one reservoir, and wherein the therapeutically effective amount of nalmefene is delivered essentially by one actuation of the self injector device.

In some embodiments, the device can be actuatable with one hand.

In some embodiments, said device is filled with said pharmaceutical formulation using sterile filling.

In some embodiments, said pharmaceutical formulation is storage-stable for about twelve months at about 25° C. and about 60% relative humidity.

Also provided are devices as recited in any of the preceding embodiments for use in the prevention of an opioid overdose symptom selected from: respiratory depression, altered level consciousness, miotic pupils, cardiovascular depression, hypoxemia, acute lung injury, aspiration pneumonia, sedation, and hypotension.

Also provided are devices as recited in any of the preceding embodiments for use in the prevention of respiratory depression induced by opioids.

In some embodiments, said therapeutically effective amount of an opioid antagonist, e.g., nalmefene, is self-administered by an individual with little or no training.

In some embodiments, said therapeutic formulation is administered to an individual in need of treatment by an untrained individual.

### Methods and Indications

Also provided are methods of using the opioid antagonists disclosed herein, and the formulations and devices comprising them, for preventing (prophylaxis of) an opioid intoxication or overdose, or symptoms thereof, caused by incidental exposure of a subject to an opioid agonist. In some embodiments, the opioid antagonist is naloxone, naltrexone, or nalmefene.

Accordingly, also provided herein are methods for preventing (prophylaxis) an opioid overdose or symptoms thereof, caused by incidental exposure of a subject to an opioid agonist, comprising administering to a subject in need thereof a therapeutically effective amount of an opioid antagonist selected from nalmefene and pharmaceutically acceptable salts thereof, wherein said therapeutically effective amount is about 3.0-10.0 mg of naloxone and/or an equivalent amount of a salt and/or solvate thereof, e.g., nalmefene hydrochloride. The naloxone can be self-administered using an auto-injector device that requires little or no prior training, or can be administered to an individual in need of such treatment by another individual with little or no training. The naloxone can be administered in the thigh, e.g. at anterolateral aspect of the thigh, through clothing if necessary, or in other muscles including the deltoid and gluteus maximus.

Accordingly, also provided herein are methods for preventing (prophylaxis) an opioid overdose or symptoms thereof, caused by incidental exposure of a subject to an opioid agonist, comprising administering to a subject in need thereof a therapeutically effective amount of an opioid antagonist selected from naltrexone and pharmaceutically acceptable salts thereof, wherein said therapeutically effective amount is about 2.0 mg to about 8.0 mg of naltrexone and/or an equivalent amount of a salt and/or solvate thereof, e.g., naltrexone hydrochloride. The naltrexone can be self-administered using an auto-injector device that requires little or no prior training, or can be administered to an individual in need of such treatment by another individual with little or no training. The naltrexone can be administered in the thigh, e.g. at anterolateral aspect of the thigh, through clothing if necessary, or in other muscles including the deltoid and gluteus maximus.

Accordingly, also provided herein are methods for preventing (prophylaxis) an opioid overdose or symptoms thereof, caused by incidental exposure of a subject to an opioid agonist, comprising administering to a subject in need thereof a therapeutically effective amount of an opioid antagonist selected from nalmefene and pharmaceutically acceptable salts thereof, wherein said therapeutically effective amount is about 2.0 mg to about 2.0 mg of nalmefene and/or an equivalent amount of a salt and/or solvate thereof, e.g., nalmefene hydrochloride. The nalmefene can be self-administered using an auto-injector device that requires little or no prior training, or can be administered to an individual in need of such treatment by another individual with little or no training. The nalmefene can be administered in the thigh, e.g. at anterolateral aspect of the thigh, through clothing if necessary, or in other muscles including the deltoid and gluteus maximus.

In some embodiments, said subject is an opioid overdose subject caused by incidental exposure of said subject to an opioid agonist or a suspected exposure to an opioid agonist.

In some embodiments, said therapeutically effective amount of an opioid antagonist is delivered by the exposed individual, members of the military, law enforcement, professional security personnel, personnel providing emergency medical services, or an untrained individual when exposure to an opioid agonist is anticipated; e.g. by law enforcement personnel searching a building during a drug raid.

In some embodiments, the opioid overdose symptom caused by incidental exposure of a subject to an opioid agonist is selected from: respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting.

In some embodiments, said opioid overdose symptom caused by incidental exposure of said subject to an opioid agonist is selected from: respiratory depression, central nervous system depression, and cardiovascular depression. In some embodiments, the symptoms caused by incidental exposure of a subject to an opioid agonist is chosen from respiratory depression and central nervous system depression. In some embodiments, said opioid overdose symptom caused by incidental exposure of said subject to an opioid agonist is respiratory depression induced by opioids.

In some embodiments, said subject exhibits any of unresponsiveness to stimulus, unconsciousness, stopped or very slow breathing; erratic or stopped pulse, choking or gurgling sounds, blue or purple fingernails or lips, slack or limp muscle tone, contracted pupils, and vomiting.

In some embodiments, the opioid antagonist, e.g. naloxone, naltrexone, or nalmefene, is provided to treat opioid overdose or symptom thereof as any of the pharmaceutical formulations for parenteral administration (parenteral pharmaceutical formulations) disclosed herein. In some embodiments, the opioid antagonist, e.g. naloxone, naltrexone, or nalmefene, is provided to treat opioid overdose or symptom thereof in any of the auto-injector devices disclosed herein, comprising said formulations.

In some embodiments, the parenteral formulation is administered prior to incidental exposure to an opioid agonist. The parenteral formulation can be administered anywhere from 5 minute to 6 hours before exposure to an opioid agonist. In some embodiments, the parenteral formulation is administered between about 5 minute and about 10 minutes prior to incidental exposure to an opioid agonist. In some embodiments, the parenteral pharmaceutical formulation is administered between about 10 minutes and about 20 minutes prior to incidental exposure to an opioid agonist. In some embodiments, the parenteral pharmaceutical formulation is administered between about 20 minutes and about 40 minutes prior to incidental exposure to an opioid agonist. In some embodiments, the parenteral pharmaceutical formulation is administered between about 40 minutes and about 60 minutes prior to incidental exposure to an opioid agonist. In some embodiments, the parenteral pharmaceutical formulation is administered between about 60 minutes and about 90 minutes prior to incidental exposure to an opioid agonist. In some embodiments, the parenteral pharmaceutical formulation is administered between about 90 minutes and about 120 minutes prior to incidental exposure to an opioid agonist. In some embodiments, the parenteral pharmaceutical formulation can be administered between about 120 minutes and 360 minutes prior to incidental exposure to an opioid agonist. In some embodiments, the parenteral pharmaceutical formulation is administered contemporaneously if incidental exposure to an opioid agonist is suspected. In some embodiments, the parenteral pharmaceutical formulation is administered as quickly as possible following incidental exposure to an opioid agonist.

In some embodiments, the plasma concentration versus time curve of naloxone, naltrexone, or nalmefene in the subject, following administration of naloxone, naltrexone, or nalmefene via the devices, formulations, and methods disclosed herein, has a Tₘₐₓ of less than 30 minutes. In some embodiments, the plasma concentration versus time curve of naloxone, naltrexone, or nalmefene in the subject has a Tₘₐₓ of less than 25 minutes. In some embodiments, the plasma concentration versus time curve of naloxone, naltrexone, or nalmefene in the subject has a Tₘₐₓ of less than 20 minutes. In some embodiments, the plasma concentration versus time curve of naloxone, naltrexone, or nalmefene in the subject has a Tₘₐₓ of less than 15 minutes. In some embodiments, the plasma concentration versus time curve of naloxone, naltrexone, or nalmefene in the subject has a Tₘₐₓ of less than 10 minutes.

In some embodiments, delivery of the therapeutically effective amount of naloxone, naltrexone, or nalmefene to the subject via the devices, formulations, and methods disclosed herein, provides occupancy at Tₘₐₓ of naloxone, naltrexone, or nalmefene at opioid receptors in the respiratory control center of the subject of greater than about 90%. In some embodiments, delivery of the therapeutically effective amount of naloxone, naltrexone, or nalmefene to the subject, provides occupancy at Tₘₐₓ of naloxone, naltrexone, or nalmefene at the opioid receptors in the respiratory control center of the subject of greater than about 95%. In some embodiments, delivery of the therapeutically effective amount of naloxone, naltrexone, or nalmefene to the subject, provides occupancy at Tₘₐₓ of naloxone, naltrexone, or nalmefene at the opioid receptors in the respiratory control center of the subject of greater than about 99%.

In some embodiments, the subject is free from opioid-induced respiratory depression for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 14 hours, at least about 16 hours, at least about 18 hours, at least about 20 hours, at least about 22 hours, or at least about 24 hours following treatment comprising delivery of the therapeutically effective amount of naloxone, naltrexone, or nalmefene via the devices, formulations, and methods disclosed herein. In some embodiments, the subject is free from opioid-induced respiratory depression for at least about 1 hour to at least about 15 hours, at least about 3 hours to at least about 15 hours, at least about 3 hours to at least about 12 hours, at least about 3 hours to at least about 10 hours, or at least about 3 hours to at least about 8 hours following treatment comprising delivery of the therapeutically effective amount of naloxone, naltrexone, or nalmefene via the devices, formulations, and methods disclosed herein.

Also provided are embodiments wherein any embodiment above may be combined with any one or more of these embodiments, provided the combination is not mutually exclusive.

Also provided are the devices, pharmaceutical formulations, kits, and methods of treatment described herein for use in the treatment of an opioid overdose symptom caused by incidental exposure of said subject to an opioid agonist selected from: respiratory depression, altered level consciousness, miotic pupils, cardiovascular depression, hypoxemia, acute lung injury, aspiration pneumonia, sedation, and hypotension. Also provided are the devices, pharmaceutical formulations, kits, and methods of treatment described herein for use in the reversal of respiratory depression induced by opioids.

Also provided are devices, pharmaceutical formulations, and kits for, and methods for preventing (prophylaxis) an opioid overdose or symptoms thereof, caused by incidental exposure of a subject to an opioid agonist, comprising self-administration using an auto-injector device in need thereof a therapeutically effective amount of the opioid antagonist naloxone and a pharmaceutically acceptable salt thereof, wherein the therapeutically effective amount is equivalent to about 3.0-10.0 mg of naloxone hydrochloride. In some embodiments, the subject is not breathing and another individual administers the victim naloxone using this auto-injector device. This device can be used with little or no training.

Also provided are devices, pharmaceutical formulations, and kits for, and methods for preventing (prophylaxis) an opioid overdose or symptoms thereof, caused by incidental exposure of a subject to an opioid agonist, comprising self-administration using an auto-injector device in need thereof a therapeutically effective amount of the opioid antagonist naltrexone and a pharmaceutically acceptable salt thereof, wherein the therapeutically effective amount is equivalent to about 0.5 to about 8.0 mg of naltrexone hydrochloride. In some embodiments, the subject is not breathing and another individual administers the victim nalmefene using this auto-injector device. This device can be used with little or no training.

Also provided are devices, pharmaceutical formulations, and kits for, and methods for preventing (prophylaxis) an opioid overdose or symptoms thereof, caused by incidental exposure of a subject to an opioid agonist, comprising self-administration using an auto-injector device in need thereof a therapeutically effective amount of the opioid antagonist nalmefene and a pharmaceutically acceptable salt thereof, wherein the therapeutically effective amount is equivalent to about 0.5 to about 2.0 mg of nalmefene hydrochloride. In some embodiments, the subject is not breathing and another individual administers the victim nalmefene using this auto-injector device. This device can be used with little or no training.

In some embodiments, naloxone is the only pharmaceutically active compound in pharmaceutical formulation. In some embodiments, naloxone is naloxone hydrochloride. In some embodiments, naloxone is anhydrous naloxone hydrochloride. In some embodiments, the pharmaceutical formulation comprises a solution of naloxone hydrochloride. In some embodiments, the injection is accomplished using an auto-injection device described herein.

In some embodiments, naltrexone is the only pharmaceutically active compound in pharmaceutical formulation. In some embodiments, naltrexone is naltrexone hydrochloride. In some embodiments, naltrexone is anhydrous naltrexone hydrochloride. In some embodiments, the pharmaceutical formulation comprises a solution of naltrexone hydrochloride. In some embodiments, the injection is accomplished using an auto-injection device described herein.

In some embodiments, nalmefene is the only pharmaceutically active compound in pharmaceutical formulation. In some embodiments, nalmefene is nalmefene hydrochloride. In some embodiments, nalmefene is anhydrous nalmefene hydrochloride. In some embodiments, the pharmaceutical formulation comprises a solution of nalmefene hydrochloride. In some embodiments, the injection is accomplished using an auto-injection device described herein.

Also provided are methods, pharmaceutical formulations, and devices for preventing (prophylaxis) an opioid overdose or symptoms thereof, caused by incidental exposure of a subject to an opioid agonist, comprising self-administering using an auto-injector device a therapeutically effective amount of the opioid antagonist naloxone and pharmaceutically acceptable salt thereof, wherein the therapeutically effective amount is equivalent to about 3.0 to about 10 mg of naloxone hydrochloride and/or an equivalent amount of a salt and/or solvate thereof.

Also provided are methods, pharmaceutical formulations, and devices for preventing (prophylaxis) an opioid overdose or symptoms thereof, caused by incidental exposure of a subject to an opioid agonist, comprising self-administering using an auto-injector device a therapeutically effective amount of an opioid antagonist selected from naltrexone and a pharmaceutically acceptable salt thereof, wherein the therapeutically effective amount is equivalent to about 0.5 to about 8.0 mg of naltrexone hydrochloride and/or an equivalent amount of a salt and/or solvate thereof.

Also provided are methods, pharmaceutical formulations, and devices for preventing (prophylaxis) an opioid overdose or symptoms thereof, caused by incidental exposure of a subject to an opioid agonist, comprising self-administering using an auto-injector device a therapeutically effective amount of an opioid antagonist selected from nalmefene and pharmaceutically acceptable salts thereof, wherein the therapeutically effective amount is equivalent to about 0.5 to about 2.0 mg of nalmefene hydrochloride and/or an equivalent amount of a salt and/or solvate thereof.

Also provided are devices, kits, and pharmaceutical formulations for, and methods of preventing (prophylaxis) an opioid overdose or symptoms thereof, caused by incidental exposure of a subject to an opioid agonist, comprising administering to a subject via an auto-injection device in need thereof a therapeutically effective amount of the opioid antagonist naloxone and pharmaceutically acceptable salts thereof, wherein the therapeutically effective amount is equivalent is about 3.0 to about 10 mg of naloxone and/or an equivalent amount of a salt and/or solvate thereof, e.g., naloxone hydrochloride, as provided above.

Also provided are devices, kits, and pharmaceutical formulations for, and methods of preventing (prophylaxis) an opioid overdose or symptoms thereof, caused by incidental exposure of a subject to an opioid agonist, comprising administering to a subject via an auto-injection device in need thereof a therapeutically effective amount of the opioid antagonist naltrexone and pharmaceutically acceptable salts thereof, wherein the therapeutically effective amount is equivalent to about 2.0 to about 8.0 mg of nalrexone and/or an equivalent amount of a salt and/or solvate thereof, e.g., naltrexone hydrochloride, as provided above.

Also provided are devices, kits, and pharmaceutical formulations for, and methods of preventing (prophylaxis) an opioid overdose or symptoms thereof, caused by incidental exposure of a subject to an opioid agonist, comprising administering to a subject via an auto-injection device in need thereof a therapeutically effective amount of the opioid antagonist nalmefene and pharmaceutically acceptable salts thereof, wherein the therapeutically effective amount is equivalent to about 0.5-2.0 mg of nalmefene and/or an equivalent amount of a salt and/or solvate thereof, e.g., nalmefene hydrochloride, as provided above.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

Also provided are embodiments wherein any embodiment above can be combined with any one or more of these embodiments, provided the combination is not mutually exclusive. Also provided herein are uses in the treatment of indications or one or more symptoms thereof as disclosed herein, and uses in the manufacture of medicaments for the treatment of indications or one or more symptoms thereof as disclosed herein, equivalent in scope to any embodiment disclosed above, or any combination thereof that is not mutually exclusive. The methods and uses may employ any of the devices disclosed herein, or any combination thereof that is not mutually exclusive, or any of the pharmaceutical formulations disclosed herein, or any combination thereof that is not mutually exclusive.

### EXAMPLES

The following examples are included to demonstrate preferred embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1

### Synopsis of Protocol for Phase 1 Pharmacokinetic Evaluation of Nalmefene Administered via

### Intramuscular Injection to Healthy Volunteers

The following is a synopsis of the relevant portion of a clinical study assessing the pharmacokinetics of nalmefene conducted at Vince & Associates Clinical Research, Overland Park, Kans. The National Institute on Drug Abuse (NIDA) was the IND sponsor for this study. The drug used in this study was Nalmefene hydrochloride (nalmefene). The study was designed to have approximately 14 healthy volunteers enrolled and to have at least 10 subjects complete all study drug administrations and blood collections for PK assessments. If less than 10 subjects completed the study using the first cohort of 14, additional subjects were screened and enrolled until there were a total of at least 10 completers.

The study determined the pharmacokinetics of nalmefene administered via an IM injection at a dose of 1.5 mg, as well as the safety and tolerability of IM nalmefene.

The pharmacokinetic parameters Cₘₐₓ, Tₘₐₓ, AUC₀₋₄ and AUC_{0-inf} of nalmefene 1M administration were determined. The LM treatment was 1.5 mg nalmefene.

The study was designed to be an inpatient, randomized, 4-period, 4-treatment, 6-sequence, crossover study involving 14 healthy volunteers. Each subject received an IM dose 1.5 mg nalmefene. Subjects stayed in the inpatient facility to complete the study and were discharged following completion of discharge procedures. Subjects were called 3 to 5 days after discharge to inquire concerning adverse events (AEs) and concomitant medications since discharge.

After obtaining informed consent, subjects were screened for eligibility to participate in the study including medical history, physical examination, clinical chemistry, coagulation markers, hematology, infectious disease serology, urinalysis, urine drug and alcohol toxicology screen, vital signs and ECG. On the day after clinic admission, subjects were administered the IN-formulated drug in randomized order with 4 days between doses; the IM dose of nalmefene was administered during the fourth (last) treatment period.

Blood was collected for nalmefene PK prior to dosing and at 2.5, 5, 10, 15, 20, 30, 45 minutes and 1, 2, 3, 4, 6, 8, 12, 16, 24, 30, 36, 48, 60, and 72 hours after study drug administration. On days of study drug administration, a 12-lead ECG were performed approximately 1 hour prior to dosing and at approximately 1 and 8 hours postdose. Vital signs were measured pre-dose and approximately 0.5, 1, 2, and 8 hours postdose. On dosing days, the order of assessments was ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 15-minute period before the nominal time of blood collections. At screening, admission, discharge, and follow-up, ECG and vital signs were checked once per day. Vital signs were also checked approximately 24, 48, and 72 hours after study drug administration. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

The criteria for inclusion and exclusion in this study as well as the protocol for safety assessment is provided in detail in the examples below.

The study drugs and design were as follows: cGMP nalmefene was obtained from Rusan Pharma Ltd. The study drug was supplied to the pharmacy at the study site. A detailed description for formulating the study drug was provided to the pharmacist. The formulation was 1.0 mg nalmefene HCl/mL normal saline for injection.

The IM formulation was given as 1.5 mL in the contralateral arm from where the blood samples were obtained.

For pharmacokinetics (PK) assessments, blood was collected in sodium heparin containing tubes prior to dosing and 2.5, 5, 10, 15, 20, 30, 45 minutes and 1, 2, 4, 6, 8, 12, 16, 24, 30, 36, 48, and 72 hours after study drugs administration. Plasma was stored at ≤−60° C. until assayed. Nalmefene plasma concentrations were determined by liquid chromatography with tandem mass spectrometry.

The analysis plan was as follows: Cmax, AUC0-t, AUC0-inf, and Tmax of nalmefene were calculated.

Within an analysis of variance (ANOVA) framework, comparisons of ln-transformed PK parameters (dose normalized Cmax and AUC) were performed using a mixed effects model where sequence, period, and treatment were the independent factors. The 90% confidence interval for the ratio of the geometric least squares means of Cmax and AUC was constructed for comparison of the three IN formulations to the IM formulation. These 90% confidence intervals were obtained by exponentiation of the 90% confidence intervals for the difference between the least squares means based upon an ln scale.

AEs were coded using the most recent version of the Medical Dictionary for Regulatory Activities (MedDRA) preferred terms and were grouped by system, organ, class (SOC) designation. The severity, frequency, and relationship of AEs to study drugs were presented by preferred term by SOC grouping. Separate summaries were provided for the 4 study periods: after the administration of each dose of study drug up until the time of the next dose of study drug or clinic discharge. Listings of each individual AE including start date, stop date, severity, relationship, outcome, and duration was provided. Vital signs, ECG, and clinical laboratory parameters were presented as summary statistics.

On the day of study drug administration, a 12-lead ECG was performed approximately 1 hour prior to dosing and 1 and 8 hours postdose. Vital signs were measured predose and approximately 0.5, 1, 2, and 8 hours postdose. The order of assessments was ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 15-minute period before the nominal time of blood collections. At screening, admission, discharge, and follow-up, ECG and vital signs were checked. Vital signs were also checked once a day after dosing. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

### Subject Selection and Screening

Subjects were healthy volunteers who resided at the clinical site for the study period and fulfilled the following inclusion and exclusion criteria.

Inclusion Criteria:

Subjects were included if they fulfill the following inclusion criteria:

Males and females 18 to 55 years of age, inclusive;

Provided written informed consent;

BMI ranging from 18 to 30 kg/m², inclusive;

Adequate venous access;

No clinically significant concurrent medical conditions determined by medical history, physical examination, clinical laboratory examination, vital signs, and 12-lead ECG;

Male subjects agreed to use an acceptable method of contraception with female partners as well as not to donate sperm throughout the study and for 90 days after the last study drug administration. Female subjects of childbearing potential agreed to use an acceptable method of birth control throughout the study and for 30 days after the last study drug administration. Oral contraceptives were prohibited;

Agreed not to ingest alcohol, drinks containing xanthine >500 mg/day (e.g., Coca Cola®, coffee, tea, etc.), or grapefruit/grapefruit juice or participate in strenuous exercise 72 hours prior to admission through the last blood draw of the study.

Exclusion Criteria: Subjects were excluded if they had any of the following criteria:

Any IN conditions including abnormal nasal anatomy, nasal symptoms (i.e., blocked and/or runny nose, nasal polyps, etc.), or having a product sprayed into the nasal cavity prior to drug administration, or failed test for sense of smell;

Been administered an investigational drug within 30 days prior to Day −1;

Taken prescribed or over-the-counter medications, dietary supplements, herbal products, vitamins, or use of opioid analgesics for pain relief within 14 days of Day −1;

Positive urine drug test for alcohol, opioids, cocaine, amphetamine, methamphetamine, benzodiazepines, THC, barbiturates, or methadone at screening or admission;

Previous or current opioid, alcohol, or other drug dependence (excluding nicotine and caffeine), based on medical history;

Subject consumed greater than 20 cigarettes per day on average, in the month prior to screening, or were unable to abstain from smoking (or use of any nicotine-containing substance) for at least one hour prior to and 2 hours after IN dosing;

Systolic blood pressure (BP) less than 90 mmHg or greater than 140 mmHg; diastolic BP less than 55 mmHg or greater than 90 mmHg; respiratory rate less than 8 respirations per minute (rpm) or greater than 20 rpm;

On standard 12-lead ECG, a QTcF interval >440 msec for males and >450 msec for females;

Significant acute or chronic medical disease in the judgment of the investigator;

A likely need for concomitant treatment medication during the study;

Donated or received blood or underwent plasma or platelet apheresis within the 60 days prior to Day −1;

Female who is pregnant, breast feeding, or plans to become pregnant during the study period or within 30 days after the last drug administration;

Positive test for HBsAg, HCVAb, or HIVAb at screening;

Current or recent (within 7 days prior to screening) upper respiratory tract infection;

Abnormal liver function test (ALT, AST, total bilirubin) >1.5 times upper limit of normal.

### Study Drugs

Study Drug Source and Description: Nalmefene's systematic name is 17-cyclopropylmethyl-4,5α-epoxy-6-methylenemorphinan-3,14-diol. NIDA supplied cGMP-grade nalmefene HCl (manufactured by Rusan Pharma, Ltd.) to the pharmacy at the clinical site. The pharmacy prepared the nalmefene HCl solution for IM administration at a strength of 1.0 mg/mL using sterile saline for injection, USP. The IM solution was tested for sterility, pyrogenicity, and other quality control tests before release for administration.

An aliquot of each dosing solution was sent to Research Triangle Institute for the determination of nalmefene concentrations.

A detailed description for formulating the study drugs was provided by the pharmacist in a separate document.

Study Drug Administration: Subjects were dosed with at least 5 minutes intervals between subjects. 1.5 mL of the 1.0 mg/mL nalmefene solution was administered in the arm contralateral from the one used for blood collection. Subjects were given the IM formulation between 08:00 am and 10:00 am.

Study Drug Accountability: The investigator maintained a log of all study drug administration to subjects throughout the trial. In addition, the study drugs were inventoried and audited against administration records.

Used/Unused Supplies: At the end of the study, the unused study drugs were inventoried. If the study drug was lost or damaged, its disposition was documented. Unused study drugs were retained at the clinical site pending instructions by NIDA for disposition at the end of the study.

### Study Procedures

Subject Screening Assessments: Screening of subjects to establish eligibility occurred initially before clinic entry and was completed at the time of clinic admission. Assessments performed during screening included collection of demographic information, medical history, a 12-lead ECG, physical examination, height, weight, BMI, nasal passage examination, sense of smell, and measurement of vital signs (heart rate, blood pressure, respiratory rate, temperature). The subjects were asked about alcohol and consumption of caffeine containing beverages or food (e.g., coffee, tea, chocolate, cola and drinks such as Red Bull®) and cigarette smoking to assure eligibility. Urine was collected for medical urinalysis and a urine toxicology screen. Blood was collected for hematology, chemistries, coagulation markers, a serum pregnancy test (if female), and viral serology (HIVAb, HBsAg, and HCVAb). Subjects were asked about prescription and over-the-counter medication, dietary supplements, herbal products, and vitamins use or recent use of opioid analgesics for pain relief in the 30 days prior to the start of screening. This information was updated throughout the screening process up to the time of clinic admission.

An eligibility checklist was provided and was reviewed at the completion of the outpatient screening assessments. If the subject remained eligible, he/she was scheduled to undergo clinic admission procedures and final eligibility assessments.

Up to 18 subjects (14 to be enrolled and 4 backups) underwent clinic admission procedures and were assessed for final eligibility on Study Day −1. Fourteen eligible subjects, including at least 6 females, were randomized.

Admission screening procedures occurred on Study Day −1. The following assessments were performed to determine eligibility: Update on medication use since the last visit; update of medical history (new diseases or conditions since last visit); physical examination and nasal passage examination; test for sense of smell; 12-lead ECG; vital signs [sitting (5 minutes) blood pressure, heart rate, respiration rate, and temperature] (may be repeated twice); chemistries, coagulation, hematology, serum pregnancy test, and urinalysis; urine drug and alcohol toxicology screen (both must be negative to be eligible); and review of eligibility checklist.

Study Drug Administration, PK Sample Collection, and Safety Monitoring: All subjects received the IM dose on the same day. At approximately 1 hour prior to dosing, ECG, blood pressure, heart rate, and respiration rate was measured and the time was recorded. At approximately 1 and 8 hours after dosing, the ECG was repeated and the time was recorded. Vital signs including sitting (after 5 minutes) heart rate, blood pressure and respiration rate were measured predose and approximately 0.5 (reclining position), 1, 2, and 8 hours after each administration.

The measurement at 0.5 hours postdose was taken in the reclining position as the subject was to remain reclining for 1 hour post administration. A physician was present during dosing and for at least 5 minutes after administration. A clinical staff member observed the subject for at least 1 hour after dosing.

Blood was collected in 4-mL sodium heparin tubes for PK analysis prior to dosing and at 2.5, 5, 10, 15, 20, 30, 45 minutes and 1, 2, 3, 4, 6, 8, 12, 16, 24, 30, 36, 48, 60, and 72 hours after study drugs administration. On dosing days, the order of assessments was ECG, vital signs, then PK blood collection when scheduled at the same nominal times. The target time of the PK blood collection was considered the most critical. If the collection time was more than ±1 minute from the scheduled time for the first 60 minutes of collections or more than ±5 minutes for the scheduled time points thereafter, this was considered a protocol deviation. ECG and vital signs were collected within the 15-minute period before the nominal time of blood collections.

A total of 352 mL of blood in 88 samples were collected for PK analysis. Another 48 mL (males) to 63 mL (female) of blood was collected for clinical laboratory assessments during the trial. The estimated total volume of blood that was collected was 400 mL for males and 415 mL for females.

Dietary and Other Restrictions: Subjects were required to abstain from nicotine and products containing caffeine or other xanthines (e.g., coffee, tea, chocolate, cola, and drinks such as Red Bull™) for at least 1 hour prior to and 2 hours after dosing. No alcohol consumption was permitted throughout the inpatient study period. Subjects were to abstain from smoking (or use of any nicotine-containing substance) for at least 1 hour prior to and 2 hours after dosing. Subjects fasted from midnight the day before dosing sessions until at least one hour after the study drugs were administered. Water was provided ad libitum. A standardized diet was provided for all meals for the duration of the inpatient portion of the study. Breakfast was provided approximately 1 hour after dosing, lunch approximately 4 hours after dosing, dinner approximately 10 hours after dosing, and a snack approximately 13 hours after dosing.

Study Drug Discontinuation: Subjects were closely monitored while inpatient before and after drug administration. Vital signs, ECG measurements, and AE reports were used to determine the safety of nalmefene administrations and the appropriateness for administering the next dose. Vital signs needed to be within acceptable limits before nalmefene was administered.

On the day that the study drug was administered, the following were the vital signs criteria that needed to be met before dosing (with subject sitting at least 5 minutes before obtaining measures): Systolic blood pressure: 140 mmHg or less and equal to or greater than 90 mmHg; Diastolic blood pressure: 90 mmHg or less and equal to or greater than 55 mmHg; Heart rate: 100 beats per minute (bpm) or less and equal to or greater than 40 bpm; Respiratory rate: 20 respirations per minute (rpm) or less and equal to or greater than 8 rpm. Vital signs could be repeated once. In addition, a clinically significant abnormal ECG at any time after clinic admission necessitated study drugs discontinuation.

Concomitant Medication Use: Subjects were not permitted to take prescription or over-the-counter medications, oral contraceptives, herbal products, dietary supplements, or vitamins during the inpatient period; however, medical treatment was not denied in the judgment of the Investigator.

Clinic Discharge: On the day of discharge from the clinic, whether at the end of the study or if a subject withdraws prematurely, the following assessments were conducted: Concomitant medications; AEs; Chemistry, coagulation markers, hematology, urinalysis, serum pregnancy test; Physical exam and nasal passage exam; Test for sense of smell; Urine drug and alcohol toxicology screen; ECG; Vital Signs.

Subjects received a telephone call 3 to 5 days after clinic discharge to inquire as to whether they had any AEs or used any medications since discharge. If any clinically significant AEs were ongoing at the time of the phone call, they were followed until resolution or stabilized.

Volunteer Discontinuation:

Early Termination for an Individual Subject: The criteria for terminating study participation for a single subject were: systolic blood pressure >180 mmHg, diastolic blood pressure >110 mmHg, respiratory rate <8 or >24 rpm confirmed by repeat; significant arrhythmia defined as >6 beats of supraventricular tachycardia or ≥3 beats of ventricular tachycardia; QTcF interval >500 msec; reported significant nausea or abdominal pain; reported significant chest pain or dyspnea; subject confusion, seizures or seizure like behavior, agitation or inability to cooperate; subject requested to leave the experiment or was unwilling or unable to cooperate in carrying out the assigned protocol procedures.

If stopping criteria were exceeded, subjects were closely observed and treated as necessary to assure return to their normal baseline state before being discharged from the clinic or transferred to another treatment facility. If more than 2 subjects showed a similar pattern of excessive cardiovascular or behavioral change or a pattern of change from baseline after drug administration not readily explainable by other factors, the study was halted.

Subject Discontinuation for Protocol Violations: Subjects were excluded or discharged if their behavior was disruptive, noncompliant with study procedures, or otherwise inconsistent with remaining in the clinic.

Subject Withdrawal: Any subject who wished to withdraw from the study on his/her own accord and for whatever reason was entitled to do so without obligation. The Investigator documented a subject's reason(s) for withdrawal from the study and indicated whether he/she thought this was related to study drugs. Any procedures/examinations planned for the subject on completion of the study were conducted as soon as possible following withdrawal. A subject was considered lost to follow-up if he/she did not respond to 2 telephone calls. Subjects who withdrew for medical reasons continued to be followed up by the Investigator or other physicians as appropriate.

Risks and Discomfort: Most of the safety data regarding the use of nalmefene came from subjects using opioid drugs, in which nalmefene may precipitate opioid withdrawal. All subjects were queried about opioid drug abuse and dependence and tested for opioid drug use (including methadone) prior to the start of the study to minimize the chances for withdrawal symptoms occurring during the study. Withdrawal is characterized by nausea, chills, myalgia, dysphoria, abdominal cramps, and joint pain. Common adverse reactions of nalmefene with an incidence greater than 1% are nausea, vomiting, tachycardia, hypertension, postoperative pain, fever, dizziness, headache, chills, hypotension, and vasodilation. Adverse events of nalmefene with an incidence less than 1% include bradycardia, arrhythmia, diarrhea, dry mouth, somnolence, depression, agitation, nervousness, tremor, confusion, withdrawal syndrome, myoclonus, pharyngitis, pruritus, and urinary retention.

### Assessment Methods

Adverse Events: Reports of AEs were elicited by a verbal probe (e.g., “How are you feeling?”) administered starting after clinic admission. Any events spontaneously reported by the subject or observed by the investigative staff were also recorded. AEs were assessed for severity and relationship to the study drugs in accordance with the criteria described below.

Clinical Chemistries: Clinical chemistries included total protein, albumin, blood urea nitrogen, creatinine, alkaline phosphatase, alanine aminotransferase, aspartate aminotransferase, total bilirubin, sodium, potassium, chloride, CO₂, calcium, glucose, and total cholesterol. The laboratory performing these assessments were either directly regulated by CAP or CLIA or indirectly according to CLIA guidelines. The laboratory needed to provide a copy of current certification.

Coagulation Markers: Coagulation markers including prothrombin time and activated partial thromboplastin time were performed. The laboratory performing these assessments were either directly regulated by CAP or CLIA or indirectly according to CLIA guidelines. The laboratory needed to provide a copy of current certification.

Demographics: Age, gender, race/ethnicity, date of birth, and date and time of signing the informed consent were collected.

ECG: Twelve-lead ECGs were performed according to standard procedures. Subjects were supine for at least 5 minutes prior to obtaining ECGs. The results were reviewed by the investigator or study physician for interpretation. The investigator could consult a board-certified cardiologist, if necessary. QT interval was corrected (QTcF) using the Fridericia formula (Fridericia L. S., Acta Medial Scandinavica. 1920; 53:469-586).

Eligibility Checklist: An eligibility checklist that included the inclusion/exclusion criteria was used at the end of out-patient screening and reviewed on the day of clinic admission to assure eligibility to participate in the study.

Hematology: A complete blood cell count including the following was performed: hemoglobin, hematocrit, red blood cells, total white blood cells; and automated differential and platelet count. The laboratory performing these assessments was either directly regulated by CAP or CLIA or indirectly according to CLIA guidelines. The laboratory needed to provide a copy of current certification.

Infectious Disease Serology: Serology for HIVAb, HBsAg, and HCVAb was performed at screening. Only those subjects with negative tests for these viruses were eligible for enrollment into the study. The results of the HIVAB testing were retained by the study site under confidential restriction; information regarding this test result at no time become part of the study database.

Study Drug Administration Record: Administration of the study drug was reported on a Study Drug Administration Record case report form (CRF) including the date and time of administration of study drug. The dose, route, and time of administration was recorded. The nostril used for dose administration was recorded. If problems occurred, these were also recorded.

Medical History: A medical history was taken on all potential study subjects to assure medical fitness including questions about current and past opioid use, abuse, and dependence and recent smoking history. Women were asked about their choice of method for birth control. Subjects were queried about recent alcohol and xanthine containing products consumption to assure eligibility.

Nalmefene Plasma Levels: Blood was obtained via direct venipuncture or through an IV catheter in the forearm of the arm which was left in place through the collection period or longer, if possible. Four milliliters of blood were collected into a sodium heparin-containing Vacutainer® tube at each time point. Plasma nalmefene concentrations were determined using a sensitive and specific validated liquid chromatography-tandem mass spectrometry method at a bioanalytical laboratory.

Physical Examination: A physical exam of the oral cavity, head, eyes, ears, nose, neck, and throat, heart, lungs, abdomen, liver, extremities, skin, neurologic, lymph nodes, and psychiatric (mental status), and general appearance was performed by a physician during screening. Height and weight were recorded at screening. BMI was calculated to determine if the subject was eligible for the study. During screening and after each dose, the nasal passage was examined by a physician for evidence of irritation (erythema, edema, and erosion). The nasal passage examination was performed after blood sample collections when the timing of collection was the same.

Prior and Concomitant Medication Use: Prescription and over-the-counter medications, herbal products, dietary supplements, and vitamins used in the 30 days prior to the start of screening and up to the day of clinic admission were recorded as prior medications. In addition, any medications taken by the subject, except study drugs, whether they were prescription or over-the-counter medications, herbal products, dietary supplements, and vitamins from the day of clinic admission until the last day of the study were considered concomitant medications. Oral contraceptives were not permitted. No concomitant medications were permitted except if the physician prescribed a medication to treat an AE or other concurrent illness. All medication used during the prior and concomitant medication use periods were recorded on the Prior and Concomitant Medications CRF.

Pregnancy Test: An FDA-cleared qualitative serum pregnancy test that evaluates human β-chorionic gonadotropin was performed by the local clinical laboratory to test all female subjects.

Urinalysis: A medical urinalysis for specific gravity, glucose, bilirubin, ketones, blood, pH, protein, nitrite, and leukocyte esterase was performed by the local clinical laboratory.

Vital Signs: Vital signs to be collected included sitting (for at least 5 minutes) blood pressure, heart rate, and respiration rate before and after dosing with an exception for 30 minutes after IN administration, which was collected in the reclining position. Sitting (for at least 5 minutes) blood pressure, heart rate, respiration rate, and temperature were checked at all other times.

Urine Drug and Alcohol Toxicology Screen: A urine toxicology screen for alcohol, opioids, cocaine, amphetamine, methamphetamine, benzodiazepines, barbiturates, THC, and methadone was performed by the local clinical laboratory.

Clinic Discharge/Final Subject Disposition: The subject disposition CRF documented all data relevant to subject discharge from the clinic: reason for discharge (i.e., completion of inpatient portion of the study, or early termination from the study) and date of discharge.

### Regulatory and Reporting Requirements

Good Clinical Practices: This study was conducted in accordance with the most current version of the International Conference on Harmonization Guidance Document E6(R1): Good Clinical Practices: Consolidated Guideline. An Operations Manual was provided to all investigational sites as a study quality assurance tool.

FDA Form 1572: The Principal Investigator signed a Statement of Investigator (FDA Form 1572) prior to initiating this study. The Form 1572 was updated as needed.

IRB Approval: Prior to initiating the study, the Principal Investigator obtained written approval from the IRB of record to conduct the study. If changes to the study protocol became necessary, protocol amendments were submitted in writing to the local IRB by the site Principal Investigator for IRB approval prior to implementation. In addition, NIDA and the local IRB approved all advertising materials used for subject recruitment and any educational materials given to the subject. Progress reports were submitted to the local IRB annually or at a frequency requested by the IRB.

Informed Consent: All potential subjects for the study were given a current copy of the informed consent form to read and take home. All aspects of the study were explained in lay language. All study subjects were given a copy of the signed informed consent.

Drug Accountability: Upon receipt, the investigator or designee was responsible for taking inventory of the study drugs. A record of this inventory was kept and usage was documented. Any unused or expired study drug was disposed according to directions provided by the Sponsor.

### Outside Monitoring:

Medical Monitor: A medical monitor was appointed for the study. The medical monitor was available for making recommendations to the investigator and the sponsor on the severity of any serious adverse events (SAEs), the relatedness to the study interventions, and for determining if the SAE should be reported to the FDA in a 7 or 15 day expedited report or an annual report. The medical monitor was also responsible for tracking and assessing trends in the AEs reported. If the medical monitor and investigator did not concur on SAE evaluations, both opinions were reported to the FDA.

Clinical Monitors: All investigators allowed representatives of the Sponsor to periodically monitor, at mutually convenient times during and after the study, all case report forms (CRFs) and corresponding source documents for each subject. These monitoring visits provided the Sponsor with the opportunity to evaluate the progress of the study and to inform the Sponsor of potential problems. The monitors assured that submitted data were accurate and in agreement with source documentation; verified that the study drugs were properly stored and accounted for, verified that subjects' consent for study participation had been properly obtained and documented, confirmed that research subjects entered into the study met inclusion and exclusion criteria, and assured that all essential documentation required by GCP guidelines were appropriately filed.

Monitors conducted a study initiation visit prior to the start of the study. At this visit, they assured that proper study-related documentation existed, assisted in training investigators and other site personnel in study procedures and GCP guidelines, confirmed receipt of study supplies, and assured that acceptable facilities and staff were available to conduct the study.

Routine monitoring visits by NIDA's representatives were scheduled at appropriate intervals. At these visits, the monitors verified that study procedures were being conducted according to the protocol guidelines and reviewed AEs and SAEs and drug accountability. At the end of the study, they advise on storage of study records and disposal of unused study drugs according to directions provided by the Sponsor.

Adverse Events Reporting: In accordance with FDA reporting requirements, all AEs occurring during the clinical trial were collected, documented, and reported by the Investigator or sub-investigators according to the procedure described below. The occurrence of AEs was assessed starting when the subject received the first dose of study drugs, then daily during the inpatient portion of the study until clinic release, and at the final follow-up telephone contact.

An AE is defined as any reaction, side effect, or untoward event that occurs during the clinical trial, whether the event is considered related to the study drug or clinically significant. For this study, events reported by the subject, as well as clinically significant abnormal findings on physical examination, vital signs, ECG, or laboratory evaluation were recorded on the AE CRF. A new illness, symptom, sign or clinically significant clinical laboratory abnormality or worsening of a pre-existing condition or abnormality was considered an AE. Stable chronic conditions, such as arthritis, which were present prior to clinical trial entry and did not worsen were not considered AEs.

All AEs, recorded during the inpatient portion of the study regardless of severity, were followed by study physicians until satisfactory resolution. AEs were required to be reported up to the date of final follow-up following hospital discharge. At the follow-up visit, AEs were recorded and followed; they were followed to resolution only if they were serious, or if the study physician assessed them to be clinically significant.

Serious Adverse Events: Each adverse event or reaction was classified by a study physician as being serious or non-serious. Based on the seriousness of the adverse event or reaction, appropriate reporting procedures were followed. The Code of Federal Regulations Title 21 part 312.32 and International Conference on Harmonization (ICH) Guideline for Industry: Clinical Safety Data Management: Definitions and Standards for Expedited Reporting, ICH-E2A March 1995, as implemented by the U.S. Food and Drug Administration, defines a serious adverse event (SAE) or serious adverse drug experience as any untoward medical occurrence at any dose that: (i) results in death; (ii) is life-threatening; (NOTE: The term “life-threatening” in the definition of “serious” refers to an event in which the subject was at risk of death at the time of the event; it does not refer to an event which hypothetically might have caused death if it were more severe.); (iii) requires inpatient hospitalization or prolongation of existing hospitalization; (iv) results in persistent or significant disability/incapacity; or (v) is a congenital anomaly/birth defect.

In addition, important medical events that may not result in death, be life-threatening, or require hospitalization were considered a serious adverse drug reaction, when based on appropriate medical judgment that may jeopardize the subject and may require medical or surgical intervention to prevent one of the outcomes listed in the above definition.

An unexpected AE is one that is not described with respect to nature, severity, or frequency in the current product package insert.

Reporting of AEs and SAES is described in below. There were serious consequences including ultimately, criminal and/or civil penalties for sponsors who failed to comply with FDA regulations governing the reporting of SAES. The Investigator in this study had the responsibility of promptly reporting all SAES to the designated Medical Monitor at NIDA in order that NIDA can comply with these regulations.

If a study subject withdrew from the study or if the Investigator decided to discontinue the subject from the study because of an SAE, the subject was required to have appropriate follow-up medical monitoring including, if necessary, hospitalization. Monitoring continued until the problem prompting hospitalization had resolved or stabilized with no further change expected or was discovered to be clearly unrelated to study medication or progresses to death.

### Analytical Plan

Study Endpoints: The primary endpoints of the study were the pharmacokinetic parameters Cₘₐₓ, Tₘₐₓ, AUC₀₋₄, and AUC_{0-inf} of nalmefene administered as 3 IN treatments and as the IM treatment: 3 mg nalmefene IN, 3 mg nalmefene plus 0.25% Intravail IN, 1.5 mg nalmefene IN, and 1.5 mg nalmefene IM.

The secondary endpoints of the study were to determine the secondary pharmacokinetic parameters and adverse events (AEs), vital signs (heart rate, sitting blood pressure; and respiration rate), electrocardiogram (ECG), clinical laboratory changes and nasal irritation (erythema, edema, and erosion) following administration of nalmefene.

Study Populations:

Safety Population: The safety population included all subjects who receive at least one administration of the study drug.

PK Evaluable Population: The evaluable population included all subjects who completed at least one treatment with sufficient sampling time points to derive meaningful PK parameters.

Sample Size: This pilot study was designed to obtain information regarding the PK of IN nalmefene under the conditions of this study. The number of subjects was deemed appropriate for this type of study.

Descriptive Statistics: Summaries of the demographics (N, age, weight, height, BMI, gender, race, and ethnicity) were provided. Demographics were also calculated for each gender (N, age, weight, height, BMI, race, and ethnicity).

PK Data Analyses: Individual plasma concentrations over time were tabulated and summarized. The following summary statistics were presented: N, arithmetic mean, SD of the arithmetic mean, median, minimum and maximum. Plasma concentration versus time curves (individual and mean) was presented.

The pharmacokinetic parameters (Cₘₐₓ, Tₘₐₓ, AUC₀₋ₜ, AUC_{0-∞}, t_{1/2}, λz, and apparent clearance (CL/F) (Table 1) were calculated using noncompartmental methods with a PK software program (Phoenix WinNonlin version 6.3 or higher, Pharsight Corp, Mountain View, Calif.) or equivalent.

TABLE 1
PK parameters of nalmefene.

| Parameter | Definition |
|---|---|
| Cₘₐₓ | Maximum plasma concentration, observed by inspection of individual study |
| | participant plots of plasma concentration versus time. |
| Cₘₐₓ/Dose | Cₘₐₓ adjusted for the nominal administered dose. |
| Tₘₐₓ | Time of maximum observed concentration, obtained directly from the |
| | observed concentration versus time data. |
| AUC₀₋ₜ | The area under the concentration-time curve from time 0 (pre-dose) to the |
| | time of last quantifiable concentration, calculated by the linear-up/log-down |
| | trapezoidal method. |
| AUC₀₋ₜ/Dose | AUC₀₋ₜ adjusted for the nominal administered dose. |
| AUC_{0-inf} | Area under the concentration-time curve from time 0 extrapolated to infinity, |
| | calculated as AUC₀₋ₜ + Cₗₐₛₜ/λz, where Cₗₐₛₜ is the observed last quantifiable |
| | concentration. |
| AUC_{0-inf}/Dose | AUC_{0-inf} adjusted for the nominal administered dose. |
| AUC %_{Extrap} | The percentage of AUC_{0-inf} obtained by extrapolation, calculated as |
| | [(AUC_{0-inf} − AUC₀₋ₜ)/AUC_{0-inf}] * 100. |
| λz | λz is the terminal-phase elimination rate constant, estimated by linear |
| | regression of logarithmically-transformed concentration versus time data. |
| t½ | The terminal phase half-life for drug concentrations in plasma is calculated |
| | as: t½ = ln(2)/λz. |
| CL/F | Apparent total body clearance is calculated as CL/F = Dose/AUC_{0-inf}. |
| Relative | Ratio of dose-adjusted AUC_{inf} following IN administration relative to dose- |
| Bioavailability | adjusted AUC_{inf} following IM administration. |

Individual PK parameters were tabulated and summarized. The following summary statistics were presented for PK parameters: N, arithmetic mean, SD of the arithmetic mean, geometric mean, SD of the geometric mean, median, minimum, and maximum. Tₘₐₓ were presented as N, median, minimum, and maximum.

Statistical Analysis of PK Parameters: Comparisons of Cₘₐₓ, AUC₀₋ₜ, and AUC_{0-inf} administration of nalmefene were calculated. The relative bioavailability of nalmefene following the 3 IN administrations was determined by comparing the dose-adjusted AUC_{0-inf} after IN administration to that of the IM formulation.

Within an ANOVA framework, comparisons of ln-transformed PK parameters (Cₘₐₓ and AUC) were performed using a mixed effects model where sequence, period, and treatment were the independent factors. The 90% confidence interval for the ratio of the geometric least squares means of Cₘₐₓ and AUC parameters was constructed for comparison of the three IN formulations to the IM formulation. These 90% confidence intervals were obtained by exponentiation of the 90% confidence intervals for the difference between the least squares means based upon an ln scale.

Safety Data Analyses: Clinically significant values of systolic and diastolic blood pressure, heart rate, temperature, and respiration rate were presented. Clinically significant ECG changes were presented by dosing session.

Adverse Events: AEs were coded using the most recent version of the Medical Dictionary of Regulatory Activities (MedDRA) preferred terms and were grouped by system, organ, class (SOC) designation. The severity, frequency, and relationship of AEs to study drugs were presented by preferred term by SOC grouping. Separate summaries were provided for 4 study periods: after each dose, up to the point of the next dose of clinic discharge. Listings of each individual AE including start date, stop date, severity, relationship, outcome, and duration were provided.

Clinical Laboratory Parameters: Clinically significant concentrations of analytes were presented for each group by dosing session.

Missing Data: Missing data were not to be imputed. The numbers of data points reflected in summary statistics were indicated by presenting the number of observations.

### Data Management and Case Report Forms

Data management activities and statistical analytical support were coordinated through the Data Management Center.

Data Collection: Data were collected at the study sites on source documents, which were transcribed at the site into case report forms (CRFs). The CRFs were supplied by the Data Management Center. CRFs were to be completed on an ongoing basis during the study. The medical chart and the source documents were the source of verification of data. Completed CRFs were collected by clinical monitors after monitoring against the source documents on a regular basis throughout the trial. The Investigator was responsible for maintaining accurate, complete and up-to-date records for each subject. The Investigator was also responsible for maintaining any source documentation related to the study, including clinical laboratory data, ECG tracings, etc.

Case Report Form Completion: Electronic CRFs (eCRF) were provided for each subject. The subject identifiers and actual date (and time, if applicable) of each assessment were entered in the eCRFs. The final, completed eCRF for each subject were signed and dated by the Investigator on the appropriate CRF page to signify that he/she had reviewed it and certified it to be complete and accurate.

Data Editing and Control: Data received at the Data Management Center were reviewed, verified and edited prior to being entered into the main study database. If incomplete or inaccurate data were found, a data clarification request was forwarded to the clinical site for a response. The site resolved data inconsistencies and errors prior to returning data to the Data Management Center. All corrections and changes to the data were reviewed prior to being entered into the main study database. Data entry into the database utilized a single-data entry procedure with 100% quality control verification of all data entered into the database.

The Investigator agreed to routine data audits by Sponsor's appointed monitors to assure that data submitted on the appropriate forms agreed with source documents at the sites. They also verified that investigational agents had been properly stored and accounted for, subject informed consent for study participation had been obtained and documented in the subject's progress notes, all essential documents in accordance with GCP guidelines were on file, and sites were conducting the study according to the research protocol. Any inconsistencies were resolved, and any changes to the data forms were made using established Data Management Center procedures.

Data Processing: A database was constructed from the eCRFs that captured each item of data from each CRF. The data were validated both manually and electronically. The database underwent 100% quality assurance audit before locking and release for statistical analysis.

All AE information was entered into the main study database from the AE CRF. AEs were coded using both the preferred term and system, organ, class designation using the most current version of MedDRA at the time of database closure.

Study Documentation and Records Retention: Study documentation included all eCRFs, data correction forms, workbooks, source documents, monitoring logs and appointment schedules, sponsor and investigator correspondence and regulatory documents (e.g., signed protocol and amendments, IRB correspondence and approved consent form and signed informed consent form, statement of Investigator form, and clinical supplies receipt and distribution records).

Source documents included all original recordings of observations or notations of clinical activities and all reports and records necessary for the evaluation and reconstruction of the clinical research study. Accordingly, source documents included, but were not limited to, laboratory reports, ECG tracings, X-rays, radiologist reports, subject diaries, biopsy reports, ultrasound photographs, subject progress notes, hospital charts or pharmacy records and any other similar reports or records of any procedure performed in accordance with the protocol.

Whenever possible, the original recording of an observation was retained as the source document; however, a photocopy was acceptable if it was a clear, legible, and exact duplication of the original document.

Government agency regulations and directives required that the investigator retain all study documentation pertaining to the conduct of a clinical trial. These documents are to be kept for a minimum of 2 years after discontinuation of the IND or two years after the approval of an NDA.

### Confidentiality:

Confidentiality of Data: Attention was drawn to the regulations promulgated by the Food and Drug Administration (FDA) under the Freedom of Information Act providing, in part, that proprietary information furnished to clinical investigators and IRBs be kept confidential by the FDA only if maintained in confidence by the clinical investigator and IRB.

By signing this protocol, the Investigator affirmed to NIDA that information furnished to the investigator by NIDA will be maintained in confidence and such information will be divulged to the IRB, expert committee; affiliated institution; and employees only under an appropriate understanding of confidentiality with such board or committee, affiliated institution and employees.

Confidentiality of Subject Records: To maintain subject confidentiality, all laboratory specimens, eCRFs, reports and other records were identified by a coded study subject number and alpha code only. Research and clinical records were stored in a locked cabinet. Only research staff, the NIDA monitoring contractor, and NIDA program officials had access to the records. Subject information was not released without written permission, except as necessary for monitoring by the FDA, the NIDA monitoring contractor, or NIDA personnel.

### Evaluation and Reporting Adverse Events and Serious Adverse Events

General Procedure: AEs were recorded after the first dose of study drug was administered. AEs were reported on an AE CRF. The severity of the event following the guidance below was reported. The relatedness of the event to the study drug administration according to the guidance below was reported.

Severity of events: Mild: awareness of symptom, but easily tolerated; Moderate: discomfort enough to cause interference with usual activity; Severe: incapacitating with inability to work or do usual activity.

Relatedness of events: The study physician was responsible for defining, in his/her best judgment, the relationship of the AE/SAE to the study drug. The degree of certainty for which the AE/SAE was attributed to the study drug or alternative causes (e.g. natural history of the underlying disease, concomitant therapies, etc.) was determined by how well the experience was understood in terms of one or more of the following:

Exposure: is there evidence that the subject was exposed to the study drug? Timing of the administration of study drug: did the AE/SAE follow in a reasonable temporal sequence from administration of the study drug?

Consistency with study drug safety profile: known pharmacology and toxicology of the study drug in animals and man; reaction of similar nature having been previously described with the study drug.

Alternative explanations for the adverse event such as concomitant medications, concurrent illness, non-medicinal therapies, diagnostic tests, procedures or other confounding findings.

Response to discontinuation of the study drug: terms and definitions to be used in assessing the study drug relationship to the AE/SAE were:

Unknown: this category was to be used only if the cause of the AE/SAE was not possible to determine;

Definitely Not Related: the subject did not receive study drug, the temporal sequence of the AE/SAE onset relative to administration of the study drug was not reasonable, or there was another obvious cause of the AE/SAE.

Unlikely Related: there was evidence of exposure to the study drug or there was another more likely cause of the AE/SAE.

Possibly Related: there was evidence of exposure to the study drug, the temporal sequence of the AE/SAE onset relative to administration of the study drug was reasonable, but the AE/SAE could have been due to another equally likely cause.

Probably Related: there was evidence of exposure to the study drug, the temporal sequence of the AE/SAE onset relative to administration of the study drug was reasonable, and the AE/SAE was more likely explained by the study drug than by any other cause.

Definitely Related: there was evidence of exposure to the study drug, the temporal sequence of the AE/SAE onset relative to administration of the study drug was reasonable, the AE/SAE was more likely explained by the study drug than by any other cause, and the AE/SAE showed a pattern consistent with previous knowledge of the study drug or study drug class.

Specific instructions—laboratory/ECG adverse event: A laboratory or ECG AE is any clinically significant worsening in a test variable that occurs during the study, whether considered to be study drug related. For each such change, information requested on date of test, severity, likelihood of a relationship to study drug, change in study drug dosage due to the AE, and treatment required were provided.

All laboratory AEs were specified as an increased or decreased test result (e.g. “increased glucose,” “decreased potassium”) or as a term that implies an abnormality (e.g., hyperkalemia, azotemia, hypokalemia, or bradycardia). Any abnormal laboratory value that was considered not clinically significant was recorded as such on the clinical laboratory report CRF along with a comment providing justification for that determination.

### Serious Adverse Event and Unexpected Adverse Event Reporting:

24-hour Reporting Requirements: Any serious adverse event, including death due to any cause, which occurred to any subject from the time of admission through discharge whether related to the study drug, was reported within 24 hours to the NIDA Medical Monitor and the NIDA Project Officer via email.

Follow-up of all adverse events/serious adverse events: All adverse medical events were followed until they were resolved, or until all attempts to determine the resolution of the AE/SAE were exhausted. This required an extended hospitalization period or a change in status from outpatient to inpatient. All treatments, outcomes and information regarding whether the subject was referred to their Primary Care Provider for additional follow-up was recorded in the source document. All serious and unexpected AEs occurring up to the final safety evaluation were reported. All follow-up Day 18-20 AEs were recorded and followed to resolution only if they were serious, or if the study physician assessed them to be clinically significant.

The investigator was required to provide the Medical Monitor and the IND Sponsor with all relevant follow-up information necessary to facilitate a thorough understanding of the event and judgment regarding the relationship to the study drug.

Reporting to the FDA: The IND Sponsor was required to report SAEs to the FDA:
in 7 days if the SAE was unexpected (or, if expected, unusually serious or rarely seen), life-threatening or lethal, and at least possibly related to the study drug, with a follow-up written report in 8 days;
in 15 days if the SAE was unexpected (or, if expected, unusually serious or rarely seen), but not immediately life-threatening; and
in an annual report in all other cases.

### Summary of PK Parameters

Table 2, below provides the mean (% CV) plasma concentrations of nalmefene following a single intramuscular administration of nalmefene to healthy subjects. The coefficient of variability, expressed as a percent (% CV) is provided within parenthesis.

TABLE 2
Mean (% CV) Plasma Concentrations of Nalmefene Following a Single
Intramuscular Administration of Nalmefene to Healthy Subjects

1.5 mg IM

| Hour | Mean | (SD) |
|---|---|---|
| 0.0 | 0.0 | (NC) |
| 0.04 | 0.0 | (NC) |
| 0.08 | 0.457 | (120) |
| 0.17 | 1.01 | (57.6) |
| 0.25 | 1.43 | (70.8) |
| 0.33 | 1.33 | (47.5) |
| 0.50 | 1.19 | (31.7) |
| 0.75 | 1.14 | (25.2) |
| 1.0 | 1.08 | (28.0) |
| 2.0 | 1.03 | (37.3) |
| 3.0 | 0.878 | (35.4) |
| 4.0 | 0.798 | (31.5) |
| 6.0 | 0.688 | (27.0) |
| 8.0 | 0.603 | (31.7) |
| 12.0 | 0.470 | (44.5) |
| 16.0 | 0.298 | (74.2) |
| 24.0 | 0.128 | (134) |
| 30.0 | 0.0740 | (164) |
| 36.0 | 0.0 | (NC) |
| 48.0 | 0.0 | (NC) |
| 60.0 | 0.0 | (NC) |
| 72.0 | 0.0 | (NC) |

N = 10-14
lower limit of quantitation (LLOQ) = 0.2 ng/mL

TABLE 3
Mean Pharmacokinetics of Nalmefene Following a Single Intramuscular
Administration of Nalmefene to Healthy Subjects.

1.5 mg IM^{b}

| Parameter (units) | Mean | (% CV) |
|---|---|---|
| Cₘₐₓ (ng/mL) | 1.67 | (50.6) |
| Cₘₐₓ/D (ng/mL/mg) | 1.12 | (50.6) |
| Tₘₐₓ (h)^{c} | 0.33 | (0.25, 8.00) |
| AUC₀₋ₜ (ng · h/mL) | 11.6 | (38.4) |
| AUC_{0-inf} (ng · h/mL) | 14.8 | (35.6) |
| AUC_{0-inf}/D (ng · h/mL/mg) | 9.86 | (35.6) |
| AUCₑₓₜᵣₐₚ (%) | 22.6 | (45.3) |
| Lambda z (h⁻¹) | 0.094 | (35.5) |
| Half-life (h) | 8.45 | (42.7) |
| CL/F (L/h) | 115 | (36.8) |

a: N = 14
^{b}N = 13
^{c}Median (minimum, maximum)

### Formulations of Intramuscular (IM) Nalmefene

The following tables set forth examples of formulations of nalmefene for IM administration for the treatments disclosed herein. Table 4 sets forth simple aqueous solution formulations such as those used in the experiment above, to be dispensed in increments of about 100 μL.

TABLE 4
Aqueous Solutions of Nalmefene.

| Ex. | Nalmefene HCl, dose (mg) | μL per dose | Conc., mg/mL |
|---|---|---|---|
| 1 | 1.5 | 150 | 10 |
| 2 | 1.0 | 100 | 10 |
| 3 | 1.25 | 125 | 10 |
| 4 | 1.75 | 175 | 10 |
| 5 | 2.0 | 200 | 10 |

Table 5 sets forth formulations for 1M administration in an aqueous solution including excipients such as an isotonicity agent and a stabilizing agent. EDTA stands for disodium edetate.

TABLE 5
Formulations for IM Administration of Nalmefene.

| Ex. | Nalmefene HCl, mg | Isotonicity Agent | Stabilizing Agent |
|---|---|---|---|
| 1a | 1.5 | NaCl 0.74% | n/a |
| 1b | 1.5 | NaCl 0.74% | EDTA 0.2% |
| 2a | 1.0 | NaCl 0.74% | n/a |
| 2b | 1.0 | NaCl 0.74% | EDTA 0.2% |
| 3a | 1.25 | NaCl 0.74% | n/a |
| 3b | 1.25 | NaCl 0.74% | EDTA 0.2% |
| 4a | 1.75 | NaCl 0.74% | n/a |
| 4b | 1.75 | NaCl 0.74% | EDTA 0.2% |
| 5a | 2.0 | NaCl 0.74% | n/a |
| 5b | 2.0 | NaCl 0.74% | EDTA 0.2% |

### Example 2

### Synopsis of Protocol for Phase 1 Pharmacokinetic Evaluation of Naltrexone Administered Via Intramuscular Injection to Healthy Volunteers

Study Goals. The purpose of this clinical study was to determine the pharmacokinetics of a 2-mg intramuscular dose of naltrexone. To that end, the study's primary endpoints were the pharmacokinetic parameters (Cₘₐₓ, Tₘₐₓ, AUC₀₋ₜ, and AUC_{0-inf}) produced by the IM dose of naltrexone. Secondary endpoints included adverse events (AEs), vital signs (heart rate, sitting blood pressure, and respiration rate), electrocardiogram (ECG), clinical laboratory changes and nasal irritation using the nasal irritation scale.

Study design. Fourteen healthy volunteers were enrolled and completed the drug administration and blood collection for PK assessments. This was an in-patient open-label, crossover study involving approximately 14 healthy volunteers. Each subject received the naltrexone treatment: 2 mg intramuscular (IM). Subjects stayed in the in-patient facility for 13 days to complete the entire study. Subjects were called 3 to 5 days after discharge to inquire concerning AEs and concomitant medications since discharge. Informed consent was obtained from all subjects, and all were screened for eligibility to participate in the study, including medical history, physical examination, clinical chemistry, coagulation markers, hematology, infectious disease serology, urinalysis, urine drug and alcohol toxicology screen, vital signs and ECG.

On the day after clinic admission, subjects were administered study drug. Blood was collected for analysis prior to dosing and approximately 2.5, 5, 10, 15, 20, 30, 45, 60 minutes and 2, 3, 4, 6, 8, 12, 16, 24, 30, 36, and 48 hours after study drug administration. On days of study drug administration, a 12-lead ECG was performed approximately 1 hour prior to dosing and at approximately 1 and 4 hours post-dose. Vital signs were measured pre-dose and about 1 and 4 hours post-dose.

On dosing days, the order of assessments was ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 10-minute period before the nominal time of blood collections. At screening, admission, and discharge, ECG, and vital signs were checked once per day. Vital signs were also checked once on the day after naltrexone administration. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

Inclusion and exclusion criteria. 1. Males and females 18 to 55 years of age, inclusive were included in this study. Written informed consent was required.

Subject had to: have body mass index (BMI) ranging from 18 to 30 kg/m2, inclusive; have adequate venous access; have no clinically significant concurrent medical conditions determined by medical history, physical examination, clinical laboratory examination, vital signs, and 12-lead ECG; agree to use an acceptable method of contraception, other than oral contraceptives, throughout the study and for 90 days after the last study drug administration (30 days for women); and agree not to ingest alcohol, drinks containing xanthine >500 mg/day (e.g., Coca-Cola®, tea, coffee, etc.), or grapefruit/grapefruit juice or participate in strenuous exercise 72 hours prior to admission through the last blood draw of the study.

Exclusion criteria included: having been administered an investigational drug within 30 days prior to Day −1; having taken prescribed or over-the-counter medications, dietary supplements, herbal products, vitamins, or recent use of opioid analgesics for pain relief (within 14 days of last use of any of these products); a positive urine drug test for alcohol, opioids, cocaine, amphetamine, methamphetamine, benzodiazepines, tetrahydrocannabinol (THC), barbiturates, or methadone at screening or admission; previous or current opioid, alcohol, or other drug dependence (excluding nicotine and caffeine), based on medical history; consumption of greater than 20 cigarettes per day on average, in the month prior to screening, or would be unable to abstain from smoking (or use of any nicotine-containing substance) for at least one hour prior to and 2 hours after naltrexone dosing; systolic blood pressure less than 90 mm Hg or greater than 140 mm Hg; diastolic blood pressure less than 55 mmHg or greater than 90 mmHg; respiratory rate less than 8 respirations per minute or greater than 20 respirations per minute; on standard 12-lead ECG, a QTcF interval >440 msec for males and >450 msec for females; significant acute or chronic medical disease (investigator judgment); a likely need for concomitant medication treatment during the study; donated or received blood or underwent plasma or platelet apheresis within the 60 days prior to Day −1; female who is pregnant, breast feeding, or plans to become pregnant during the study period or within 30 days after the last naltrexone administration; positive test for hepatitis B surface antigen (HBsAg), hepatitis C virus antibody (HCVAb) or human immunodeficiency virus antibody (HIVAb) at screening; current or recent (within 7 days prior to screening) upper respiratory tract infection; and abnormal liver function test (ALT, AST, total bilirubin) >1.5 times upper limit of normal.

Study Drugs and Dosing. Naltrexone hydrochloride (HCI) was obtained from Mallinckrodt Pharmaceuticals. The IM formulation (2 mg/mL) was made by the staff pharmacist at Vince & Associates; the vehicle was sterile saline for injection. Naltrexone HCI for the IM injection was administered with a 23-g needle as a single 1-mL injection into the gluteus maximus muscle.

Naltrexone was administered at a dose of 2 mg IM.

PK Assessments. Blood (4 mL) was collected in sodium heparin containing tubes for PK analysis prior to dosing and 2.5, 5, 10, 1.5, 20, 30, 45, 60 minutes and 2, 3, 4, 6, 8, 12, 16, 24, 30, 36, and 48 hours after the start of study drug administration. Plasma was separated from whole blood and stored frozen at ≤20° C. until assayed. Naltrexone plasma concentrations were determined by liquid chromatography with tandem mass spectrometry at XenoBiotic Laboratories, Inc., Plainsboro, N.J.

Safety Assessments. Heart rate, blood pressure, and respiration rate were recorded approximately 1 hour before naltrexone dosing and approximately 1 and 4 hours after dosing. A 12-lead echocardiogram (ECG) was obtained about 1 hour before and 1 and 4 hours after the naltrexone dose. ECG and vital signs was performed within the 10-minute period before the nominal time for post-dose blood collections. Adverse events (AEs) were recorded from the start of study drug administration until clinic discharge.

Analysis. Non-compartmental PK parameters naltrexone including, Tmax, AUC0-t, and AUC0-inf, t1/2, λz, and apparent clearance was determined. Dose-adjusted values for AUCs and Cmax were calculated.

Naltrexone Results. Results are shown below in Table 6 and Table 7 and at FIG. 1.

TABLE 6
Mean (SD) concentrations of naltrexone following
a single IM administration to healthy subjects.

2.0 mg IM

| Hour | Mean | SD |
|---|---|---|
| 0 | 0 | 0 |
| 0.042 | 0.678 | (1.69) |
| 0.083 | 1.04 | (1.26) |
| 0.17 | 2.97 | (2) |
| 0.25 | 3.45 | (1.58) |
| 0.33 | 3.58 | (1.46) |
| 0.5 | 3.43 | (1.06) |
| 0.75 | 3.02 | (0.749) |
| 1 | 2.73 | (0.676) |
| 2 | 2.35 | (0.698) |
| 3 | 1.79 | (0.491) |
| 4 | 1.3 | (0.341) |
| 6 | 0.584 | (0.185) |
| 8 | 0.242 | (0.0803) |
| 12 | 0.0626 | (0.0269) |
| 16 | 0.0101 | (0.0132) |
| 24 | 0 | 0 |
| 30 | 0 | 0 |
| 36 | 0 | 0 |
| 48 | 0 | 0 |

TABLE 7
Mean (CV %) PK Parameters for Naltrexone Following
Administration to Healthy Subjects.

2.0 mg IM

| Parameter (units) | Mean | (% CV) |
|---|---|---|
| Cₘₐₓ (ng/mL) | 4.10 | (34.0) |
| Cₘₐₓ/Dose (ng/mL/mg) | 2.27 | (34.0) |
| Tₘₐₓ (h)^{b} | 0.33 | (0.17, 1.00) |
| AUC₀₋ₜ (h · ng/mL) | 12.1 | (25.5) |
| AUC₀₋ₜ/Dose (h · ng/mL/mg) | 6.71 | (25.5) |
| AUC_{0-inf} (h · ng/mL) | 12.3 | (25.6) |
| AUC_{0-inf}/Dose (h · ng/mL/mg) | 6.78 | (25.6) |
| AUCₑₓₜᵣₐₚ (%) | 1.01 | (71.7) |
| CL/F (L/h) | 154 | (19.0) |
| λ_{z} (1/h) | 0.361 | (16.8) |
| t½ (h) | 1.97 | (15.5) |
| Fᵣₑₗ | NA | NA |

^{a} N = 10
^{b}Median (minimum, maximum)

The mean plasma concentrations of naltrexone at 2.5 and 5 minutes after administration of 2 mg naltrexone IM were 0.678 ng/mL and 1.04 ng/mL, respectively.

The mean terminal phase half-life (t½) of naltrexone was 1.97 hours after IM administration.

### Formulations of Intramuscular (IM) Naltrexone

The following tables set forth examples of formulations of naltrexone for IM administration for the treatments disclosed herein. Table 8 sets forth simple aqueous solution formulations such as those used in the experiment above, to be dispensed in increments of about 300 μL-1.0 mL.

TABLE 8
Aqueous Solutions of Naltrexone.

| Ex. | Naltrexone HCl, dose (mg) | μL per dose | Conc., mg/mL |
|---|---|---|---|
| 6 | 2.0 | 400 | 5 |
| 7 | 2.5 | 400 | 6.25 |
| 8 | 3.0 | 400 | 7.5 |
| 9 | 3.5 | 400 | 8.75 |
| 10 | 4.0 | 400 | 10 |
| 11 | 4.5 | 400 | 11.25 |
| 12 | 5.0 | 400 | 12.5 |
| 13 | 5.5 | 400 | 13.75 |
| 14 | 6.0 | 400 | 15 |
| 15 | 6.5 | 400 | 16.25 |
| 16 | 7.0 | 400 | 17.5 |
| 17 | 7.5 | 400 | 18.75 |
| 18 | 8.0 | 400 | 20 |

Table 9 sets forth formulations for IM administration in an aqueous solution including excipients such as an isotonicity agent and a stabilizing agent. EDTA stands for disodium edetate.

TABLE 9
Formulations for IM Administration of Naltrexone.

| Ex. | Naltrexone HCl, mg | Isotonicity Agent | Stabilizing Agent |
|---|---|---|---|
| 6a | 2.0 | NaCl 0.74% | n/a |
| 6b | 2.0 | NaCl 0.74% | EDTA 0.2% |
| 7a | 2.5 | NaCl 0.74% | n/a |
| 7b | 2.5 | NaCl 0.74% | EDTA 0.2% |
| 8a | 3.0 | NaCl 0.74% | n/a |
| 8b | 3.0 | NaCl 0.74% | EDTA 0.2% |
| 9a | 3.5 | NaCl 0.74% | n/a |
| 9b | 3.5 | NaCl 0.74% | EDTA 0.2% |
| 10a | 4.0 | NaCl 0.74% | n/a |
| 10b | 4.0 | NaCl 0.74% | EDTA 0.2% |
| 11a | 4.5 | NaCl 0.74% | n/a |
| 11b | 4.5 | NaCl 0.74% | EDTA 0.2% |
| 12a | 5.0 | NaCl 0.74% | n/a |
| 12b | 5.0 | NaCl 0.74% | EDTA 0.2% |
| 13a | 5.5 | NaCl 0.74% | n/a |
| 13b | 5.5 | NaCl 0.74% | EDTA 0.2% |
| 14a | 6.0 | NaCl 0.74% | n/a |
| 14b | 6.0 | NaCl 0.74% | EDTA 0.2% |
| 15a | 6.5 | NaCl 0.74% | n/a |
| 15b | 6.5 | NaCl 0.74% | EDTA 0.2% |
| 16a | 7.0 | NaCl 0.74% | n/a |
| 16b | 7.0 | NaCl 0.74% | EDTA 0.2% |
| 17a | 7.5 | NaCl 0.74% | n/a |
| 17b | 7.5 | NaCl 0.74% | EDTA 0.2% |
| 18a | 8.0 | NaCl 0.74% | n/a |
| 18b | 8.0 | NaCl 0.74% | EDTA 0.2% |

### Formulations of Intramuscular (IM) Naloxone

The following tables set forth examples of formulations of naloxone for IM administration for the treatments disclosed herein. Table 10 sets forth simple aqueous solution formulations such as those used in the experiment above, to be dispensed in increments of about 300 μL-1.0 mL.

TABLE 10
Aqueous Solutions of Naloxone.

| Ex. | Naloxone HCl, dose (mg) | mL per dose | Conc., mg/mL |
|---|---|---|---|
| 19 | 3.0 | 400 | 7.5 |
| 20 | 3.5 | 400 | 8.75 |
| 21 | 4.0 | 400 | 10 |
| 22 | 4.5 | 400 | 11.25 |
| 23 | 5.0 | 400 | 12.5 |
| 24 | 5.5 | 400 | 13.75 |
| 25 | 6.0 | 400 | 15 |
| 26 | 6.5 | 400 | 16.25 |
| 27 | 7.0 | 400 | 17.5 |
| 28 | 7.5 | 400 | 18.75 |
| 29 | 8.0 | 400 | 20 |
| 30 | 8.5 | 400 | 21.25 |
| 31 | 9.0 | 400 | 22.5 |
| 32 | 9.5 | 400 | 23.75 |
| 33 | 10.0 | 400 | 25 |

Table 9 sets forth formulations for IM administration in an aqueous solution including excipients such as an isotonicity agent and a stabilizing agent. EDTA stands for disodium edetate.

TABLE 9
Formulations for IM Administration of Naloxone.

| Ex. | Naloxone HCl, mg | Isotonicity Agent | Stabilizing Agent |
|---|---|---|---|
| 19a | 3.0 | NaCl 0.74% | n/a |
| 19b | 3.0 | NaCl 0.74% | EDTA 0.2% |
| 20a | 3.5 | NaCl 0.74% | n/a |
| 20b | 3.5 | NaCl 0.74% | EDTA 0.2% |
| 21a | 4.0 | NaCl 0.74% | n/a |
| 21b | 4.0 | NaCl 0.74% | EDTA 0.2% |
| 22a | 4.5 | NaCl 0.74% | n/a |
| 22b | 4.5 | NaCl 0.74% | EDTA 0.2% |
| 23a | 5.0 | NaCl 0.74% | n/a |
| 23b | 5.0 | NaCl 0.74% | EDTA 0.2% |
| 24a | 5.5 | NaCl 0.74% | n/a |
| 24b | 5.5 | NaCl 0.74% | EDTA 0.2% |
| 25a | 6.0 | NaCl 0.74% | n/a |
| 25b | 6.0 | NaCl 0.74% | EDTA 0.2% |
| 26a | 6.5 | NaCl 0.74% | n/a |
| 26b | 6.5 | NaCl 0.74% | EDTA 0.2% |
| 27a | 7.0 | NaCl 0.74% | n/a |
| 27b | 7.0 | NaCl 0.74% | EDTA 0.2% |
| 28a | 7.5 | NaCl 0.74% | n/a |
| 28b | 7.5 | NaCl 0.74% | EDTA 0.2% |
| 29a | 8.0 | NaCl 0.74% | n/a |
| 29b | 8.0 | NaCl 0.74% | EDTA 0.2% |
| 30a | 8.5 | NaCl 0.74% | n/a |
| 30b | 8.5 | NaCl 0.74% | EDTA 0.2% |
| 31a | 9.0 | NaCl 0.74% | n/a |
| 31b | 9.0 | NaCl 0.74% | EDTA 0.2% |
| 32a | 9.5 | NaCl 0.74% | n/a |
| 32b | 9.5 | NaCl 0.74% | EDTA 0.2% |
| 34a | 10.0 | NaCl 0.74% | n/a |
| 34b | 10.0 | NaCl 0.74% | EDTA 0.2% |

Although the present invention has been described with reference to specific details of certain embodiments thereof in the above examples, it will be understood that modifications and variations are encompassed within the spirit and scope of the invention.

1. A method for preventing opioid overdose or a symptom thereof in a subject caused by incidental exposure to an opioid agonist, comprising self-administering a parenteral injection of a pharmaceutical formulation comprising an effective amount of an opioid antagonist and/or an equivalent amount of a salt and/or solvent thereof using an auto-injection device.

2. The method of claim 1, wherein the opioid antagonist is selected from the group consisting of naloxone, naltrexone, and nalmefene.

3. The method of claim 2, wherein the pharmaceutical formulation comprises:
a) about 2.0 mg to about 8.0 mg of naltrexone, or about 0.5 mg to about 3.0 mg nalmefene, or about 3.0 to about 10.0 mg of naloxone, and/or an equivalent amount of a salt and/or solvate of any of the foregoing;
b) about 0.1 mg to about 6.0 mg of an isotonicity agent;
c) optionally a stabilizing agent; and
d) an amount of an acid or a base sufficient to achieve a pH of 3.4-4.4.

4. The method of claim 3, wherein the parenteral injection is by an intramuscular route or by a subcutaneous route.

5. The method of claims 1-3, wherein the patenteral injection is by a subcutaneous route.

6. The method of claim 3, wherein the pharmaceutical formulation comprises about 2.7 mg to about 4.5 mg of an isotonicity agent.

7. The method of claim 3, wherein the pharmaceutical formulation comprises an aqueous solution of about 300 μL to about 1.0 mL.

8. The method of claim 1, wherein the incidental exposure to opioid agonist is selected from:
a) incidental inhalation exposure by aerosolized opioid agonist; and
b) incidental transdermal or transmucosal exposure by an aerosolized or powdered form of an opioid agonist.

9. The method of claim 8, wherein the subject is a healthcare professional, personnel providing emergency medical services, law enforcement officer, (e.g., police, customs, and border patrol agents), military member, warfighter, professional security person, or an untrained individual.

10. The method of claim 8, wherein the subject is involved in the investigation or clean-up of an opioid agonist production, transport, or distribution site.

11. The method of claim 3, wherein the acid is hydrochloric acid.

12. The method of claim 3, wherein the base is sodium hydroxide.

13. The method of claim 3, wherein the isotonicity agent is sodium chloride.

14. The method of claim 1, wherein the pharmaceutical formulation is substantially free of antimicrobial preservatives.

15. The method of claim 1, wherein the pharmaceutical formulation is storage-stabile for about twelve months at about 25° C.

16. The method of claim 1, wherein the parenteral formulation is administered prior to incidental exposure to an opioid agonist.

17. The method of claim 1, wherein the parenteral formulation is administered anywhere from 5 minutes to 6 hours before exposure to an opioid agonist.

18. The method of claim 1, wherein the parenteral formulation is administered between about 5 minutes and about 10 minutes prior to incidental exposure to an opioid agonist.

19. The method of claim 1, wherein the parenteral pharmaceutical formulation is administered between about 10 minutes and about 20 minutes prior to incidental exposure to an opioid agonist.

20. The method of claim 1, wherein the pharmaceutical formulation will prevent a symptom of opioid overdose selected from the group of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting.

21. The method of claim 20, wherein the incidental exposure to an opioid agonist occurs during a drug raid or during a military operation.

22. The method of any of claims 1-20 wherein the incidental exposure to an opioid agonist occurs during a military operation.

23. A method for preventing opioid overdose or a symptom thereof in a subject caused by incidental exposure to an opioid agonist, comprising self-administering intranasally a pharmaceutical formulation comprising an effective amount of an opioid antagonist and/or an equivalent amount of a salt and/or solvent thereof.

24. The method of claim 23, wherein the opioid antagonist is selected from the group consisting of naloxone, naltrexone, and nalmefene.

25. The method of claim 23, wherein the pharmaceutical formulation comprises:
e) about 2.0 mg to about 8.0 mg of naltrexone, or about 0.5 mg to about 3.0 mg nalmefene, or about 3.0 to about 10.0 mg of naloxone, and/or an equivalent amount of a salt and/or solvate of any of the foregoing;
f) about 0.1 mg to about 6.0 mg of an isotonicity agent;
g) optionally a stabilizing agent; and
h) an amount of an acid or a base sufficient to achieve a pH of 3.4-4.4.

26. The method of claim 23, wherein the incidental exposure to opioid agonist is selected from the group consisting of:
c) incidental inhalation exposure by aerosolized opioid agonist; and
d) incidental transdermal or transmucosal exposure by an aerosolized or powdered form of an opioid agonist.

27. The method of claim 26, wherein the subject is a healthcare professional, personnel providing emergency medical services, law enforcement officer, (e.g., police, customs, and border patrol agents), military member, warfighter, professional security person, or an untrained individual.

28. The method of claim 26 wherein the subject is involved in the investigation or clean-up of an opioid agonist production, transport, or distribution site.
